(19) United States
(12) Patent Application Publication
(10) Pub. No.: US 2017/0283366 A1
(43) Pub. Date: Oct. 5, 2017

(54) Benzoquinone Derivatives For Treatment Of Cancer And Methods Of Making The Benzoquinone Derivatives

(71) Applicant: United Arab Emirates University, Al Ain (AE)

(72) Inventors: Alaa Eldin Salem, Al Ain (AE); Ibrahim Abdou, Al Ain (AE); Ismail El Haty, Al Ain (AE)

(21) Appl. No.: 15/090,457

(22) Filed: Apr. 4, 2016

(51) Int. Cl. C07C 223/06; C07D 207/06

(52) U.S. Cl. CPC C07C 223/06; C07D 207/06; C07C 2101/16

(57) ABSTRACT

The present invention benzoquinone derivatives of the formula (I):

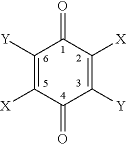

and to pharmaceutically acceptable salts or solvates thereof. In formula (I) one of X or Y is hydrogen and the other one of X or Y is 3-Trifluoro-methylaniline; 3,4,5-trifluoroaniline; 4-methoxylaniline; 4-fluoroaniline; 3,3′-Dimethyl-1,1′-Biphenyl-4,4′-diamine; 2-(pyrrolidin-l-yl)ethyl)amine; 4-trifluoromethyl-benzylamine ; 4-fluorobenzyl-amine; 3,4-dimethoxybenzylamine; or 3,5-ditrifluoromethyl-benzylamine. Compounds of formula (I) have been identified as being useful in the treatment of cancer, in particular lung, breast and pancreatic cancer. The invention relates also to a method of making the benzoquinone derivatives and to methods of treatment.

TECHNICAL FIELD

This invention relates to benzoquinone derivatives and to methods of making the benzoquinone derivatives. The invention also relates to the use of the benzoquinone derivatives in the treatment of cancer and to pharmaceutical compositions containing the benzoquinone derivatives.

### BACKGROUND TO THE INVENTION

Quinones refer to the dihydro aromatic compounds whose basic sub-unit is 1,4-benzoquinone. They are naturally found in flowering plants, bacteria, fungi, arthropods and Lichens. Quinones are antioxidant reducing agents that share in electron-transfer reactions in respiratory and photosynthesis processes. Because of their natural bright colors and biological activities, several quinone compounds have been used in traditional medicine. Since the discovery of vitamin K in 1929, many quinones have been extracted from plants or fungi, Some extracted quinones showed antibiotic, antitumor, antimalarial, anti-inflammatory, and anticoagulant properties. Nowadays, natural and synthetic quinones comprise the second largest class of anti-tumor agents currently in use. Examples include Doxorubicin, Mitoxantrone and Mitomycin C. Several mechanisms have been proposed for the anticancer effects of Quinones that include apoptosis, abrogation of cell cycles, activation of caspases, stimulating the production of reactive oxygen species (ROS), inhibition of topoisomerases I and II, activation of intracellular secondary messengers and production of free radicals to attack DNA. The anticancer activity of quinoids has been attributed to their ability to undergo a biochemical reduction by one or two electrons that are catalyzed by flavoenzymes in the organism using NADPH as an electron donor producing semiquinone radical intermediates and subsequent reactions with oxygen. These forms are believed to be responsible for most of the quinoids anticancer activity. Quinones with amino-substituted side chains have shown antitumor activity. More particularly, WO2011/126544 discloses analogues of thymoquinone for the treatment of pancreatic cancer.

Another mechanism for cancer and antitumor treatment, which is a different mechanism from that described herein above, relies on the effect which a compound may have on DNA. Prior to explaining the effect and the mechanism of the treatment, a few basic features of DNA and of chromosomes, are first described herein below.

The end regions or so-called “end caps” of chromosomes have a region of repetitive nucleotide sequences called telomeres. These telomeres protect the end of chromosomes from deterioration or from fusion with neighbouring chromosomes. More specifically, telomeres consist of so-called “tandem repeats” comprising a sequence of two or more DNA base pairs that are repeated in such a way that the repeats lie adjacent to each other on the chromosome. These tandem repeats are enriched in guanine bases and fold up under physiological conditions to form so-called “four-stranded G-quadruplex structures”. Furthermore, telomeres capping the ends of human chromosomes and promoter regions of some oncogenes (such as c-myc, k-ras and c-kit) are enriched in guanine bases. Telomeres fold up forming G-quadruplex DNA structures which inhibit telomerase enzyme which is over-expressed in cancer cells. Telomerase enzyme re-elongates telomeres causing cancer cells to continue to divide indefinitely. Formation of intramolecular G-quadruplex in the promoter region of some oncogenes seems to play an important role in regulating transcription of the corresponding gene.

It has also been hypothesized that formation of G-quadruplex DNA structures in the promoter region of some oncogenes plays an important role in regulating the transcription of the corresponding gene. Consequently, small molecules that selectively bind and stabilize G-quadruplex DNA have potential for the development of new selective and efficient anticancer therapeutic agents. Molecules which stack on the face or interact with grooves and loops of quadruplex were reported as good G-quadruplex's stabilizers.

As mentioned above, DNA sequences enriched in guanine can form high order structure called G-quadruplex DNA. This G-quadruplex DNA consists of four strands connected by eight Hoogsteen hydrogen bonds. G-quadruplex DNA formation has been reported in human telomere (chromosome's ends) and in promoter regions of some oncogenes such as c-myc, k-ras and c-kit. In normal cells the telomeres are shortened after each cell division until the apoptosis of the cell occurs at short telomere length. Telomerase enzyme re-elongates the telomeres in stem and cancer cells. Furthermore, Telomerase has been found active in almost all cancer cells and contributes to the indefinite division of tumor cells. Formation of telomere quadruplex structures has shown to inhibit telomerase enzyme in breast, prostate, lung, liver, pancreatic and colon cancers. Therefore, it was assumed to control cell transcriptions and expression.

Several compounds have been reported as G-quadruplex's stabilizers, telomerase's inhibitors and/or oncogene regulators. These included some porphyrines, porphyrazines, phtalocyanines, cyclopyrroles, telomestatin, and porphyrin TMPyP4 derivatives. 5,10,15,20-tetra [4-hydroxyl-3-9-trimethyl-ammonium) methyl-phenyl] showed higher selectivity due to the presence of four positively charged groups and four hydroxyl groups that can interact with the G-quadruplex loops and grooves. Acridines, acridones, perylene, corolone, anthraquinones and quinolone derivatives were reported to strongly interact with the G-tetrads of quadruplex DNA though π-π stacking and to duplex DNA through intercalation between duplex base pairs or grooves of DNA. The length between the aromatic central core and the positively charged nitrogen atom of the lateral side chain as well as the basicity of the system were assumed responsible for giving rise to better quadruplex stabilization by perylene.

A need exists for an antitumor agent which effectively targets telomeric G-quadruplex DNA and which stabilize G-quadruplex DNA. A need further exists for compounds having molecules with high preferential affinity to stabilize G-quadruplex structures which can inhibit/stop cancer proliferations and provide potential for developing new selective genetic anticancer therapeutic agents. Furthermore a need exists for an antitumor agent which shows a better selectivity towards G-quadruplex DNA over ct-DNA (tumor DNA circulating freely in the blood of a cancer patient). Furthermore a need exists for an antitumor agent which has a high antitumor effect and/or efficacy. Also, a need exists for an antitumor agent having improved biological potency, efficacy and selectivity. A need also exists for a synthetic benzoquinone analogue which is cost effective to synthesise, non-toxic and effective in treatment of cancer.

### SUMMARY OF INVENTION

The present invention relates to compounds of formula (I):

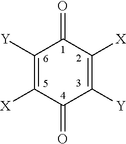

and to pharmaceutically acceptable salts or solvates thereof wherein:
one of X and Y is hydrogen and the other one of X and Y is selected from the group consisting of flouroaryl amines, biphenyl amines, amino-pyrrolidines and methoxyphenyl amines. More particularly, said other one of X and Y is selected from the group consisting of 3-Trifluoro-methylaniline; 3,4,5-trifluoroaniline; 4-methoxylaniline; 4-fluoroaniline; 3,3′-Dimethyl-1,1′-Biphenyl-4,4′-diamine; 2-(pyrrolidin-l-yl)ethyl)amine; 4-trifluoromethyl-benzylamine ; 4-fluorobenzyl-amine ; 3,4-dimethoxybenzylamine and 3,5-ditrifluoromethyl-benzylamine.

In a preferred embodiment, the compounds of formula (I) are selected from:
2,5-bis-(3-trifluoromethylanilino)-1,4-benzoquinone (BQ1);
2,5-bis-(3,4,5-trifluoroanilino)-1,4-benzoquinone (BQ2);
2,5-bis-(4-methoxylanilino)-1,4-benzoquinone (BQ3);
2,5-bis-(4-fluoroanilino)-1,4-benzoquinone (BQ4);
2,5-bis-((3,3′-Dimethyl-1,1′-Biphenyl-4-amine)-4′-amino)-1,4-benzoquinone (BQ6);
2,5-bis((2-(pyrrolidin-l-yl)ethyl)amino)-1,4-benzoquinone (BQ9);
2,5-bis-(4-trifluoromethylbenzylamino)-1,4-benzoquinone (BQ10);
2,5-bis-(4-fluorobenzylamino)-1,4-benzoquinone (BQ11);
2,5-bis-(3,4-dimethoxybenzylamino)-1,4-benzoquinone (BQ12); and
2,5-bis-(3,5-ditrifluoromethylbenzylamino)-1,4-benzoquinone (BQ14).

The compounds of formula (I) are synthetic analogues of benzoquinone.

Suitable pharmaceutically acceptable salts may include salts of acidic or basic groups present in compounds of formula (I). The compounds of formula (I) that are basic in nature are capable of forming a variety of salts with various inorganic and organic acids. The acids that may be used to prepare pharmaceutically acceptable acid addition salts of such basic compounds of formula (I) are those that form non-toxic acid addition salts. Suitable pharmaceutically acceptable salts may include acetate, benzensulfonate, benzoate, bicarbonate, bisulfate, bitartrate, borate, bromide, calcium edentate, camsylate, carbonate, chloride, clavulanate, citrate, dihydrochloride edentate, edisylate, estolate, esylate, ethylsuccinate, fumarate, gluceptate, gluconate, glutamate, glycollylarsanilate, hexylresorcinate, hydrabamine, hydrobromide, hydrochloride, iodide isothionate, lactate, lactobionate, laurate, malate, maleate, mandelate, mesylate, methylsulfate, mucate, napsylat, nitrate, oleate, oxalate, pamoate, palmitate, pantothenate, phosphate, diphosphate, diphosphate, polygalacturonate, salicylate, stearate, subacetate, succinate, tannate, tartrate, teoclate, tosylate and valerate.

The compounds of formula (I) may be synthesised by a number of synthetic routes. In one method of making the compounds of formula (I), the compounds of formula (I) can be synthesised as shown in Scheme (I).

[symbol] Scheme (I)

Compounds of formula (I) may be synthesised in a one pot reaction by reacting benzoquinone in a suitable solvent with a compound of formula (II) dissolved in the solvent, wherein the compound of formula (II) is R-NH₂, wherein R is selected from the group consisting of flouroaryl amines, biphenyl amines, amino-pyrrolidines or methoxyphenyl amines. More particularly, R is selected from the group consisting of F-Z-NH₂; CF₃-Z-NH₂, (Me)₂-Z-NH₂, MeO-Z-NH₂ or (MeO)₂-biphenyl-NH₂, where Z is selected from the group consisting of phenyl, benzyl, biphenyl, ethylpyrrolidine and biphenyl amine.

More specifically, the compound of formula (II) may be selected from the group consisting of 3-(trifluoromethyl)aniline; 3,4,5-trifluoroaniline; ρ-anisidine (4-methoxyaniline); 4-fluoroanaline; o-tolidine (4-(4-Amino-3-methylphenyl)-2-methylaniline); 1-(2-aminoethyl)pyrrolidine; 4-trifluoromethylbenzyl amine; 4-fluorobenzyl amine; 3,4-dimethoxybenzylamine veratrylamine and 3,5-bis-(trifluoromethyl)benzylamine.

The suitable solvent may be methanol.

The reaction may be achieved by introducing the compound of formula (II) in a drop by drop manner.

The reaction may occur in the presence of air at room temperature. The solvent may be concentrated and purified after the reaction. The resultant products may be crystallised after the reaction.

The compounds:
2,5-bis-(3-trifluoromethylanilino)-1,4-benzoquinone (BQ1);
2,5-bis-(3,4,5-trifluoroanilino)-1,4-benzoquinone (BQ2);
2,5-bis-(4-methoxylanilino)-1,4-benzoquinone (BQ3);
2,5-bis-(4-fluoroanilino)-1,4-benzoquinone (BQ4);
2,5-bis-((3,3′-Dimethyl-1,1′-Biphenyl-4-amine)-4′-amino)-1,4-benzoquinone (BQ6);
2,5-bis((2-(pyrrolidin-l-yl)ethyl)amino)-1,4-benzoquinone (BQ9);
2,5-bis-(4-trifluoromethylbenzylamino)-1,4-benzoquinone (BQ10);
2,5-bis-(4-fluorobenzylamino)-1,4-benzoquinone (BQ11);
2,5-bis-(3,4-dimethoxybenzylamino)-1,4-benzoquinone (BQ12); and
2,5-bis-(3,5-ditrifluoromethylbenzylamino)-1,4-benzoquinone (BQ14),
   are particularly useful in the treatment of pancreatic cancer, lung cancer, or breast cancer.

Preferably the compound 2,5-bis-(4-methoxylanilino)-1,4-benzoquinone (BQ3) is used for the treatment of pancreatic cancer, lung cancer, or breast cancer.

Preferably the compound 2,5-bis-((3,3′-Dimethyl-1,1′-Biphenyl-4-amine)-4′-amino)-1,4-benzoquinone (BQ6) is used for the treatment of pancreatic cancer, lung cancer, or breast cancer.

Preferably the compound 2,5-bis-(4-trifluoromethylbenzylamino)-1,4-benzoquinone (BQ10) is used for the treatment of pancreatic cancer, lung cancer, or breast cancer.

Preferably the compound 2,5-bis-(4-fluorobenzylamino)-1,4-benzoquinone (BQ11) is used for the treatment of pancreatic cancer, lung cancer, or breast cancer.

The compounds of formula (I) or the pharmaceutically acceptable salts or solvates thereof, have been found to bind G-quadruplex DNA, thereby to stabilise G-quadruplex DNA. Stabilisation of the G-quadruplex DNA can inhibit telomerase enzyme. Inhibition of telomerase enzyme may be beneficial in the treatment of cancer. The compounds of formula (I) or the pharmaceutically acceptable salts or solvates thereof, have been found to inhibit telomerase enzyme.

The invention also provides a method of treating cancer in a mammal, particularly a human, comprising administering to the mammal an amount of a compound of formula (I), as defined above, or pharmaceutically acceptable salts or solvates thereof. The mammal may be in need of cancer treatment. The method may be, more particularly, for the treatment of pancreatic cancer, lung cancer, or breast cancer. The compound may be administered in a therapeutically effective amount. The method may further include administering the compound of formula (I), or the pharmaceutically acceptable salts or solvates thereof, in combination with at least one suitable anti-tumor or neoplastic agent for the treatment of cancer, in particular for the treatment of pancreatic, lung, or breast cancer.

More particularly, the compounds of formula (I) or the pharmaceutically acceptable salts or solvates thereof, may be beneficial in the treatment of cancer due to the ability of the compounds of formula (I) to stabilize G-quadruplex DNA. The method may be, more particularly, for use in the treatment of pancreatic cancer, lung cancer, or breast cancer. As such, the compounds of formula (I) or the pharmaceutically acceptable salts or solvates thereof may be used in a method of stabilizing G-quadruplex DNA in a mammal, preferably in a human. The mammal or human may be in need of cancer treatment. The method may include stabilizing the G-quadruplex DNA by binding the compounds of formula (I), or the pharmaceutically acceptable salts or solvates thereof, to the G-quadruplex DNA. More specifically, the method may include stabilizing the G-quadruplex DNA by binding the compounds of formula (I) or the pharmaceutically acceptable salts or solvates thereof, to the G-quadruplex DNA.

In vitro, an affinity of the compounds of formula (I) for binding to G-quadruplex DNA are greater than an affinity of the compounds of formula (I) for binding to ct-DNA. The compounds of formula (I) may be beneficial in the treatment of cancer due to the higher affinity of the compounds of formula (I) for binding G-quadruplex DNA, when compared to the affinity of the compounds of formula (I) for binding ct-DNA.

The invention further relates to a method of stabilizing G-quadruplex DNA that may be formed in a telomere region of a mammalian chromosome, more particularly in a telomere region of a human chromosome, the method comprising binding the compound of formula (I), or a pharmaceutically acceptable salts or solvates thereof, to the G-quadruplex DNA. The mammalian chromosome or the human chromosome may be from a mammal, or human, respectively, in need of cancer treatment. The mammal or human may be in need of cancer treatment for one of pancreatic cancer, lung cancer, or breast cancer. The method may, more particularly, comprise binding a therapeutically effective amount of the compound of formula (I), or a pharmaceutically acceptable salts or solvates thereof, to the G-quadruplex DNA.

Furthermore, the compounds of formula (I), as defined above, or the pharmaceutically acceptable salts or solvates thereof, may be beneficial in the treatment of cancer due to the ability of the compounds of formula (I) to inhibit telomerase enzyme. As such, the compounds of formula (I), as defined above, or the pharmaceutically acceptable salts or solvates thereof, may be used in a method of inhibiting telomerase enzyme in a mammal, preferably a human. More particularly, the invention relates to a method of inhibit telomerase enzyme in a mammalian cell, particularly a human cell, comprising administering to the cell an amount of a compound of formula (I), as defined above, or the pharmaceutically acceptable salts or solvates thereof. The mammalian cell may be in need of cancer treatment. The method may be, more particularly, for the treatment of pancreatic cancer, lung cancer, or breast cancer. The compound may be administered in a therapeutically effective amount.

The method may comprise binding the compound of formula (I), or the pharmaceutically acceptable salts or solvates thereof, to G-quadruplex DNA that may be formed in the mammalian cell or in the human cell.

The invention relates also to the use of the compound of Formula (I), as defined above, or the pharmaceutically acceptable salts or solvates thereof, in the manufacture of a pharmaceutical composition for treating cancer. More particularly, the pharmaceutical composition may be for the treatment of pancreatic cancer, lung cancer, or breast cancer.

The invention further relates to the compounds of formula (I), as defined above, or the pharmaceutically acceptable salts or solvates thereof, in combination with at least one suitable anti-tumor or neoplastic agent for the treatment of cancer, in particular for the treatment of pancreatic, lung, or breast cancer.

The invention also relates to a pharmaceutical composition comprising a compound of formula (I) as described above, or the pharmaceutically acceptable salts or solvates thereof and a pharmaceutically acceptable diluents or carrier. The pharmaceutical composition may comprise an additional therapeutic agent. The pharmaceutical composition may be for treating a mammal, preferably a human, in need of cancer treatment. The pharmaceutical composition may be used in the treatment of pancreatic cancer, lung cancer, or breast cancer.

The term “treatment” is intended to include curing, reversing, alleviating, palliative and prophylactic treatment of the condition.

A “therapeutically effective amount” of a compound is an amount of the compound, which when administered to a subject, is sufficient to confer the intended therapeutic effect. A therapeutically effective amount can be given in one or more administrations.

Common cancers would include bladder, breast, colon, rectal, endometrial, kidney (renal cell), leukaemia, lung, melanoma, non-Hodgkin lymphoma, pancreatic, prostate, brain, skin, liver and thyroid cancers.

Patients suffering from cancer are commonly co-administered additional therapeutic agents, in particular suitable antineoplastic or anti-tumor agents. Suitable co-administrants would include:

1. Alkylating antineoplastic agents: such as cisplatin, carboplatin, oxaliplatin, mechlorethamine, cyclophosphamide, chlorambucil, and ifosfamide.
2. Plant alkaloids and terpenoids. These include:
i. vinca alkaloids such as vincristine, vinblastine, vinorelbine and vindesine
ii. podophyllotoxins such as etoposide and teniposide
iii. taxanes, such as paclitaxel, originally known as taxol, and docetaxel.
   3. Topoisomerase inhibitors:
i. type I topoisomerase inhibitors include camptothecins: irinotecan and topotecan
ii. type II inhibitors include amsacrine, etoposide, etoposide phosphate, and teniposide
   4. Cytotoxic antibiotics such as actionmycin, anthracyclins, doxorubicin, daunorubicin, valrubicin and epirubicin. Other cytoxic antibiotics include bleomycin, plicamycin and mitomycin.

Other therapeutic agents are commonly administered to patients to deal with the side effects of chemotherapy. Such agents might include anti-emetics for nausea, or agents to treat anaemia and fatigue. Other such medicaments are well known to physicians and those skilled in cancer therapy.

Such agents may be administered sequentially, simultaneously or concomitantly.

Suitable composition forms include forms suitable for oral administration such as tablets, capsule, pills, powders, sustained release formulations, solutions, and suspension, for parental injection such as sterile saline solutions, suspensions or emulsion; for topical administration such as ointments or creams; or rectal administration such as suppositories.

Exemplary parenteral administration forms include suspensions or solutions in sterile aqueous solutions, for example aqueous propylene glycol or dextrose solutions. Such dosage forms can be suitably buffered, if desired.

Suitable pharmaceutical carriers include inert diluents or fillers, water and various organic solvents. Compositions may also include additional ingredients such as flavouring, binders, and excipients. Tablets may include: disintegrates such as starch, alginic acid and complex silicates; binding agents such as sucrose, gelatine and acacia, and lubricating agents such as magnesium stearate, sodium lauryl sulphate and talc.

Solid compositions may also include soft and hard gelatin capsules. Preferred materials include lactose, milk sugars and high molecular weight polyethylene glycols.

Aqueous suspensions or elixirs may include sweetening or flavouring agents, colours and dyes, emulsifying agents, suspending agents as wells as diluents such as water, ethanol, propylene glycol, glycerin or combinations thereof.

Pharmaceutical forms suitable for the delivery of the compounds of the present invention and methods of preparing the various pharmaceutical compositions will be readily apparent to those skilled in the art. Such compositions and methods for their preparations may be found, for example in Remington's Pharmaceutical Sciences, 19^{th} Edition (Mack Publishing Company 1995).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a table showing the structure of benzoquinone compounds BQ1-BQ14 of Formula (I), in accordance with the invention;

FIG. 2 shows a table showing the names and structures of the compounds of Formula (II) used in the synthesis of the compounds BQ1-BQ14 of Formula (I);

FIG. 3 shows a schematic diagram of the one-pot synthesis mechanism of amino benzoquinone compounds (BQ1-BQ14) in accordance with the invention;

FIG. 4(a) shows a graph showing the ¹H-NMR spectrum of BQ6 in DMSO-d₆;

FIG. 4(b) shows a graph showing the ¹³C-NMR spectrum of BQ6 in DMSO-d₆;

FIG. 5 shows a graph showing the gCOSY 2D-NMR spectrum for BQ6 in DMSO-d₆;

FIG. 6 shows a graph showing the short-rang HSQC 2D-NMR spectrum for BQ6 in DMSO-d₆,

FIG. 7 shows a graph showing the long-range ¹H-¹³C gHMBC 2D-NMR spectrum for BQ6 in DMSO-d₆;

FIG. 8 shows a table showing melting temperatures (Tm), differences in melting temperatures (ΔTm), binding constants (K) and number of binding sites (n) for G-quadruplex-BQs and ct-DNA—BQs complexes;

FIGS. 9(a) to 9(n) show graphs showing UV-Vis titrations of BQ(1-14) (5×10−6 M) with telomeric G-quadruplex DNA (1.44×10−4 M) in Tris-KCl buffer, pH 7.4. The insets show Scatchard plots of the absorption data;

FIGS. 10(a) to 10(n) show graphs showing CD titrations of telomeric G-quadruplex DNA (4×10−6 M) , with BQ(1-14) in Tris-KCl buffer, pH 7.4;

FIGS. 11(a) to 11(n) show graphs showing fluorescence titrations of 5′-Fl-G-quadruplex DNA (2×10−6 M) titrated with (1×10−4 M) BQ(1-14) in Tris-KCl buffer, pH 7.4. 5′-Fl-G-quadruplex was excited at 494 nm and emitted at 518 nm;

FIG. 12 shows a table showing IC50 of BQs (in μM) for the different tested cell lines;

FIG. 13(a) shows a graph illustrating melting temperature curves for G-quaduplex and G-quadruplex-BQs complexes. A 1.0 mL of telomeric G-quadruplex (3.93×10−6 M) or 1.0 mL of telomeric G-quadruplex (3.93×10−6 M) mixed with equimolar amount of specific BQ solution in Tris-KCl-buffer, pH 7.4 were heated up in the range 25-95° C. wherein five min incubation times were applied;

FIG. 13(b) shows a graph illustrating melting temperature curves for ct-DNA and ct-DNA-BQs complexes. A 1.0 mL of ct-DNA (100 ppm, ˜10−9 M) or 1.0 ml solution of ct-DNA (100 ppm, ˜10−9 M) mixed with BQs solution (23.4×10−6 M) in Tris-KCl-buffer, pH 7.4 was heated up in the range 25-95° C. wherein five min incubation times were applied;

FIG. 14 shows a graph illustrating selectivity of the BQs towards G-quadruplex (5′-Flu-G-quad, 5×10−10 M) in presence of (0, 10, 50 and 100) folds of telomere duplex DNA in Tris-KCl buffer, pH 7.4;

FIGS. 15(a) to 15(n) show graphs illustrating inhibition of cellular viability by the BQs wherein exponentially growing human pancreatic cancer cells (L3.6Pl) were treated with vehicle (0.1% DMSO) and the indicated concentrations of the BQs, wherein viable cells were assayed as described in Materials and Methods and wherein experiments were repeated at least three times, Columns, mean; bars, SD. *Significantly different at P<0.05;

FIGS. 16(a) to 16(n) show graphs illustrating inhibition of cellular viability by the BQs wherein exponentially growing human pancreatic cancer cells (MiaPaCa-2) were treated with vehicle (0.1% DMSO) and the indicated concentrations of the BQs, wherein viable cells were assayed as described in Materials and Methods and wherein all experiments were repeated at least three times, Columns, mean; bars, SD. *Significantly different at P<0.05;

FIGS. 17(a) to 17(n) show graphs illustrating inhibition of cellular viability by the BQs, wherein exponentially growing human lung cancer cells (H1299) were treated with vehicle (0.1% DMSO) and the indicated concentrations of the BQs, wherein viable cells were assayed as described in Materials and Methods and wherein, all experiments were repeated at least three times, Columns, mean; bars, SD. *Significantly different at P<0.05;

FIGS. 18(a) to 18(n) show graphs illustrating inhibition of cellular viability by the BQs, wherein exponentially growing human breast cancer cells (MCF-7) were treated with vehicle (0.1% DMSO) and the indicated concentrations of the BQs, wherein viable cells were assayed as described in Materials and Methods, and wherein all experiments were repeated at least three times, Columns, mean; bars, SD. *Significantly different at P<0.05; and

FIGS. 19(a) to 19(n) show graphs illustrating inhibition of cellular viability by the BQs, wherein exponentially growing human prostate cancer cells (C42B) were treated with vehicle (0.1% DMSO) and the indicated concentrations of the BQs, wherein viable cells were assayed as described in Materials and Methods, and wherein all experiments were repeated at least three times. Columns, mean; bars, SD. *Significantly different at P<0.05.

### DETAILED DESCRIPTION

### Experimental

Described hereinbelow are the materials, reagents and apparatus, used:

in the synthesis of various benzoquinone derivatives (“BQ1-14”, “BQ”, “BQ derivatives” or “BQ's”), in accordance with the invention;
to examine the benzoquinone derivatives synthesized, in accordance with the invention, and the effect of the benzoquinone derivatives on cancer cell;
to investigate interactions between the BQ derivatives and telomeric G-quadruplex DNA and selectivity of the BQ derivatives towards G-quadruplex DNA over duplex DNA; and
to study binding affinity, binding stoichiometry and invitro anticancer effects of the BQ derivatives.

### Materials and Reagents

All chemicals were purchased from Sigma-Aldrich, Germany (Taufkirchen, Munchen). The following chemicals were of the highest purity grade and used without further purification; 1,4-Benzoquinone, 3-(trifluoromethyl)aniline, 3,4,5-trifluoroaniline, p-anisidine (4-methoxyaniline), 4-fluoroanaline, aniline, o-tolidine (4-(4-Amino-3-methylphenyl)-2-methylaniline), 4-(2-aminoethyl)morpholine, cyclopentylamine, 1-(2-aminoethyl)pyrrolidine, 4-trifluoromethylbenzyl amine, 4-fluorobenzyl amine, 3,4-dimethoxybenzylamine, Veratrylamine, benzylamine, 3,5-bis-(trifluoromethyl)benzylamine, methanol and Calf thymus DNA. Human telomeric DNA was purchased from Alpha DNA (Canada).

Human cancer cell lines were purchased from Hyclone Laboratories, Utah, USA. Pancreatic cancer cells (L3.6pl and MiaPaCa-2) and breast cancer cells (MCF-7) were maintained in DMEM while lung cancer cells (H1299) and prostate cancer cells (C42B) were maintained in RPMI 1640 media. All media were supplemented with antibiotics (penicillin 50 U/ml; streptomycin 50 μg/m1) and 10% fetal bovine serum (FBS, Biowest, Nouaille, France).

### Apparatus

Thin-layer chromatography (TLC) was performed on glass-silica gel plates (Silica gel, 60 F₂₅₄, Fluka) and ethyl acetate-hexane (1:1) as mobile phase. Spots were visualized under UV lamp. Column chromatography was performed on Kieselgel-S (Silica gel-S, 0.063-0.1mm). A Gallen kamp melting point apparatus was used for recording the melting points of synthesized compounds and an Euro Vector EA-3000 CHNS analyzer was used for their elemental analyses.

Infrared and NMR spectra were performed using a Thermo Nicolet model 470 FT-IR and a Varian 400 MHz FT-NMR spectrometers, respectively. IR spectra were recorded in KBr solid pellets while NMR spectra were recorded in DMSO-d₆ and CDCl₃ solutions with tetramethylsilane (TMS) as an internal reference. Mass spectra were performed using Finnigan-Trace GC 2000 (Thermo Quest, USA) equipped with a split-splitless injector, AS-3000 autosampler (Thermoelectron Corporation, USA) and a quadruple mass spectrometer (Trace-MS Finnigan) detector with mass range of 1 to 1050 a.m.u. for detecting derivatized phenoxy herbicides.

### Standard Solutions

Buffer solution (Tris-KCl buffer)

A 0.01 M Tris-KCl buffer solution-pH 7.4, was prepared by dissolving 10.00 mM of tris-hydroxymethylaminomethane hydrochloride (1.576 g), 1.00 mM Na₂EDTA (0.3722 g) and 100.00 mM KCl (7.455 g) into 1.0 L of deionized water. The pH was adjusted using glass electrode. A 1.00 mL of Tween-80 was added to the solution and shacked well.

### BQs' Solutions

Stock solutions (2×10⁻³ M) of benzoquinone derivatives were prepared in DMSO. Solutions having lower concentrations were prepared by appropriate dilution into DMSO.

### DNA Solutions

Calf Thymus DNA (ct-DNA)

Calf thymus ds-DNA (1000 μg/ml; 8×10⁻⁸ M) was prepared by dissolving 10.0 mg of DNA into 10.0 ml Tris-KCl buffer, pH 7.4, without sonication or stirring. To prevent shearing of the large genomic DNA, the solution was gently inverted overnight at 4.0° C. to completely solubilize the DNA. Solutions of DNA are stable for several months at 4.0° C. in Tris-KCl buffer pH 7-8.

### Single Stranded DNAs

Purchased synthetic nucleic acids primers with human telomere sequence; 5′-AGGGTTAGGGTTAGGGTTAGGG-3′, its fluorescein labeled 5′ primer Fl-5′-AGGGTTAGGGTTAGGGTTAGGG-3′ or its complementary strand 3′-TCCCAAT-CCCAATC-CCAATCCC-5′ were reconstituted by centrifugation for 10 min at 7000 rpm to collect DNA in the bottom of the vials. Tris-KCl buffer (2.00 ml) were added and left 2 min for rehydration then vortexed for 30 s. Reconstituted primers were kept overnight at 4.0° C. Stability of reconstituted primers is more than 6 months.

### G-Quadruplex DNA

Telomeric G-quadruplex DNA was prepared by heating gently 2.0 ml of stock single stranded 5′-AGGGTTAGGGTTAGGGTTAGGG-3′ DNA up to 95.0° C. Resultant solution was incubated at 95.0° C. for 10 min. The solution was left to cool gently down to room temperature, then kept in fridge at 4.0° C. overnight before use. A 10⁻⁵ M fluorescein labeled G-quadruplex DNA was prepared similarly using 5′-Flu-telomeric DNA.

### Hybridization of Telomeric DNA Oligonucleotides

A 1×10⁻⁴ M telomeric ds-DNA was prepared by mixing equimolar amounts of 5′-AGGGTTAGGGTTAGGGTTAGGG-3′ (268.80 μL of 7.44×10⁻⁴ M) with its complementary strand 3′-TCCCAATCCCAATCCCAATCCC-5′ (738.00 μL of 2.71×10⁻⁴ M). The solution was made up to 2000.0 μl using KCl-Tris-Cl buffer pH 7.4, vortexed for 15 s and incubated at 95.0° C. for 10.0 min then left to cool to room temperature. Resultant hybridized ds-DNA was kept in refrigerator at 4.0° C. till use.

To determine the concentrations of prepared DNA stock solutions, a 10.0 μl DNA solution was diluted using Tris-KCl-buffer solution, pH 7.4, to 1.0 ml. Resultant solution was vortexed for 15 s, followed by measuring absorbance at 260 and 280 nm. Concentration in μg/ml was calculated using the following equation:

C_{(μg/ml)}=A₂₆₀×weight per OD×dilution factor

where OD is the optical density at 260 nm. The ratio A₂₆₀/A₂₈₀ was used to estimate the purity of each oligonucleotide. Ratios ≧1.8 were considered enough to indicate high purity for synthetic and calf thymus DNAs.

### BQs' Stability and Aggregation

Stability and aggregation of BQs were studied by following changes in absorbance of 10⁻⁵ M solution of each BQ in 5.0% DMSO Tris-KCl buffer, pH 7.4 and 0.1% Tween-80 over 48 hrs.

Interactions of BQs with G-quadruplex

Interactions of the BQs with G-quadruplex DNA were studied using UV-Vis, fluorescence, fluorescence quenching and circular dichroism spectroscopies as well as melting temperature. Binding parameters such as binding constant, stoichiometry, selectivity towards G-quadruplex over duplex DNA were evaluated.

### UV-Vis Titration

Successive amounts of G-quadruplex DNA (AGGG(TTAGGG)₃ , 1.44×10⁻⁴ M) were added to 1.00 ml of each BQ (5×10⁻⁶ M) in Tris-KCl buffer, pH 7.4. Solution was shacked well after each addition, incubated for 3 min at room temperature and its absorbance was scanned in the range 200-600 nm. Titration was stopped when no change in absorbance was observed. The experiment was reversed by adding successive amounts of the each BQ (1×10⁻³ M) to 1.0 ml of 4×10⁻⁶ M G-quadruplex DNA.

### Fluorescence Quenching Titration

Binding affinity of BQs towards human telomeric G-quadruplex DNA was further confirmed using fluorescence quenching assay. Successive amounts of each BQ (1×10⁻³ M) were added to 3.00 ml of Fl-labeled G-quadruplex (Fl-5′-AGGGTTAGGGTTAGGGTTAGGG-3′) (1×10⁻⁷ M) in Tris-KCl buffer, pH 7.4. After each addition, the solution was stirred for 20 s, incubated for 3 min and scanned for fluorescence using λₘₐₓ=518 nm as excitation wavelength.

### Circular Dichroism Titration

Additional evidences on interactions of the BQs with telomeric G-quadruplex DNA were obtained using circular dichroism. Successive amounts of each BQ (1×10⁻³ M) were added to 1.0 ml telomeric G-quadruplex DNA (4×10⁻⁶ M) in Tris-KCl buffer, pH 7.4. After each addition, solution was shacked, incubated for 3 min at room temperature and scanned in the range 200-400 nm using scan speed of 50.0 nm/min and band width of 1.0 nm. Averages of at least 3 accumulation scans were considered.

### Melting Temperature Curves

Melting temperature curves for telomere G-quadruplex, ct-DNA and their BQs' adducts were constructed using CD spectral measurements. A 1.0 ml telomeric G-quadruplex (3.93×10⁻⁶ M) or ct-DNA (100.00 ppm) in Tris-KCl-buffer, pH 7.4 was heated in the range 25-95° C. applying 2.0-5.0° C. increments and using 5 min incubation time intervals. The CD spectra in the range 200-400 nm were recorded at each temperature using the scan parameters described in previous section.

BQs complexes with G-quadruplex or ct-DNA were prepared by mixing equimolar amounts of G-quadruplex (1.44×10⁻⁴ M) with BQs (1×10⁻⁴ M) or ct-DNA (1000 ppm) with BQs (1×10⁻⁴ M) solution in 1.0 ml KCl-buffer pH 7.4. Solutions were incubated for 30 min before scan.

Collected CD spectra were smoothed and baseline corrected against blank solution. Intensities of CD peaks for G-quadruplex, ct-DNA and their BQs' complexes at 293 and 282 nm were recorded. Plots of CD intensities versus temperature were constructed.

### Selectivity of the BQs Towards G-quadruplex

Selectivity of BQs towards G-quadruplex over duplex DNAs was investigated fluorometrically using duplex telomere DNA. A 3.0 ml solution that is 1×10⁻⁷ M in 5′-Fl-G-quadruplex and 1×10⁻⁷ M in BQs was mixed with 10.00, 50.00 or 100.00 folds of telomere dsDNA in Tris-KCl-buffer pH 7.4. Solutions were vortexed for 10 s, incubated for 30 min at room temperature and scanned for their fluorescence spectra in the range 500-600 nm using excitation λₘₐₓ of 494 nm.

### Stoichiometry and Binding Affinity

The molar ratio method based on measuring UV-Vis absorption was used for determining stoichiometry of G-quadruplex DNA interactions with BQs. A 1.00 ml BQs (5×10⁻⁶ M) was titrated with telomere G-quadruplex (1.44×10⁻⁴ M) in Tris-KCl buffer, pH 7.4. The solution was shacked well after each addition, incubated for 3.0 min at room temperature and scanned in the range 200-600 nm. A plot of absorbance versus molar ratio [BQs]/[DNA] was constructed.

Binding affinity of the BQs towards telomere G-quadruplex DNA were estimated using Scatchard model based on the above UV-Vis absorption titration. Scatchard plot was

$$\frac{r}{{C}_{f}}$$

constructed as versus r according to the Scatchard equation;

$$\frac{r}{{C}_{f}} nK - Kr .$$

Wherein r is the number of moles of the BQ bound to one mole of telomere G-quadruplex DNA (C_{b}/[G-quadruplex DNA], C_{f} is the free BQ's concentration, n is number of equivalent binding sites per G-quadruplex molecule and K is the binding constant. The free and bound concentrations of BQ (C_{f} and C_{b}) are calculated using C_{b}=Cₜₒₜₐₗ−C_{f}, where Cₜₒₜₐₗis the concentration of BQ at zero addition of G-quadruplex and C_{f} is calculated using C_{f}=Cₜₒₜₐₗ(1 −α). The fraction of BQ bound to G-quadruplex (α) is calculated using

$$α = \frac{{A}_{f} - A}{{A}_{f} - {A}_{b}}$$

where A_{f}, A and A_{b} are the absorbance at zero addition, after each addition and at saturation, respectively.

$$\frac{r}{{C}_{f}}$$

Linear plots of versus r give slope and intercept equals to K and n, respectively. For nonlinear plots, the modified Scatchard equation was used. Plots of r against C_{f}

$$r = \frac{{nkC}_{f}}{( 1 + {KC}_{f} )}$$

were subjected to nonlinear fitting to get the values of K and n.

### Antitumor Effect

BQs were tested for their antiprolifrative activity in vitro using MTT assay on human pancreatic cancer cells (L3.6pl and MiaPaCa-2), human lung cancer cells (II1299), human breast cancer cells (MCF-7) and human prostate cancer cells (C42B).

The cell viability test was conducted by seeding pancreatic (L3.6pl and MiaPaCa-2), lung (H1299), breast (MCF-7) and prostate (C42B) cancer cells into 96-well culture plates at a density of 3×103 cells per well. Cells were then exposed to increasing doses (0-25 μM) of BQs for 72 hrs and MTT assay was performed using thymoquinone as a positive control in all experiments. The results were plotted as means ±SD of at least three separate experiments using six determinations per experiment. Data were presented as proportional viability (%) by comparing the treated group with the untreated cells whose viability is assumed to be 100%. IC50s of the BQs were estimated from the linear plots of concentrations versus cell viabilities.

The synthesis of various benzoquinone derivatives (BQ1-14), in accordance with the invention; is described herein below by way of example.

### Synthesis of the BQ Derivatives

### General Procedure

Benzoquinone analogs were synthesized according to the following general procedure. Into a two necks flask (100 mL), 5.0 mmol benzoquinone (541 mg) were dissolved in 50.0 ml methanol (95%). Resultant solution was stirred and air bubbled followed by adding the corresponding amine solution (15.0 mmol) in methanol (5 ml) drop by drop. Stirring and air bubbling of the reaction mixture at room temperature continued overnight during which the reaction's progress was monitored by TLC. Formation of a precipitate was an indication for the reaction progress. When the reaction is completed, the solution was filtered off, and washed by methanol. The product obtained was recrystallized from methanol.

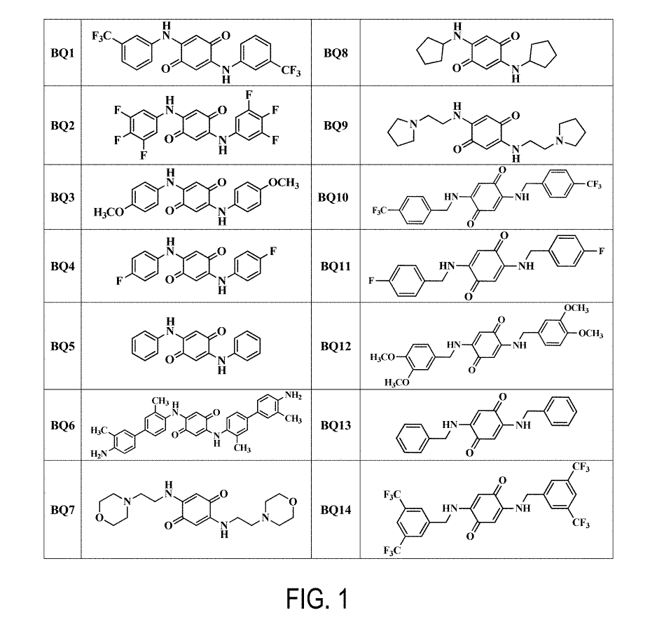

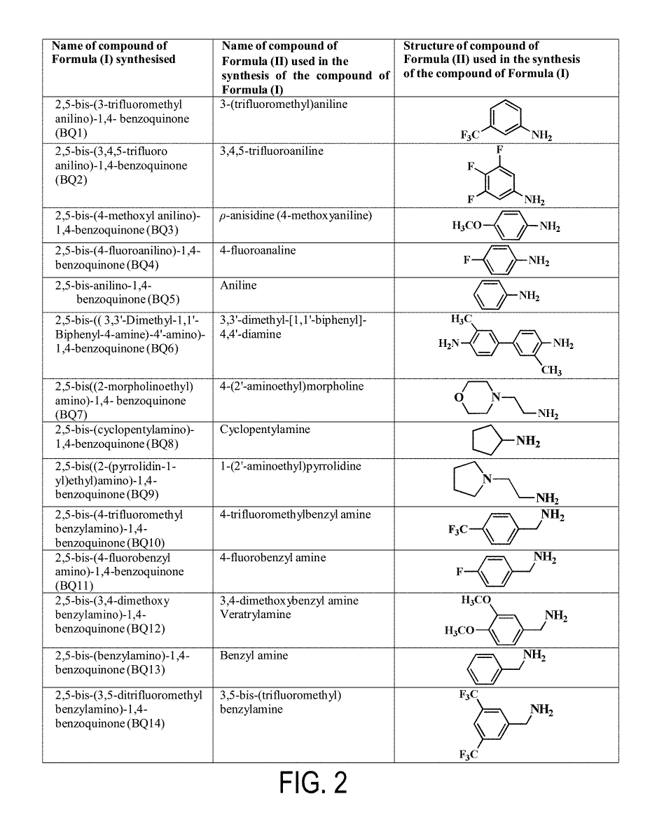

More particularly, fourteen benzoquinone analogs BQ₍₁₋₁₄₎ were synthesized by coupling benzoquinone with selected aromatic and alicyclic amines in one pot reaction under air bubbling at room temperature (Scheme 1 and FIGS. 1 to 3). More specifically, the fourteen benzoquinone analogs BQ₍₁₋₁₄₎ were synthesized by coupling benzoquinone with 3-trifluoromethyl aniline, 3,4,5-trifluoroaniline, 4-methoxyaniline, 4-fluoroaniline, aniline, 3,3′-dimethyl-[1,1′-biphenyl]-4,4′-diamine, 4-(2′-aminoethyl)morpholine, cyclopentylamine, 1-(2′-aminoethyl)pyrrolidine, 4-trifluoromethylbenzyl amine, 4-fluorobenzyl amine, 3,4-dimethoxybenzyl amine, benzyl amine and 3,5-bis-(trifluoromethyl)benzylamine The mechanism of reaction involved 3,6 or 2,5-additions of amine molecules to benzoquinone followed by rearrangement of the substituted benzoquinone into amine substituted hydrobenzoquinone. A second benzoquinone molecule oxidizes the formed substituted hydrobenzoquinone and afforded di-substituted benzoquinone derivatives. More specifically, the reactions processed by nucleophilic substitution of protons at position 2 and 5. Reaction yield was enhanced by adding excess amounts of benzoquinone and bubbling air in reaction mixture to oxidize the intermediate amino substituted hydro-benzoquinones. Refluxing at high temperature and microwave synthesis were found ineffective in improving products' yields.

Reaction's progress was monitored by TLC. The weak solubility of products in methanol protected them from further rearrangement. Structures of resulted compounds were confirmed using IR, ¹H-NMR, ¹³C-NMR, MS and elemental analyses.

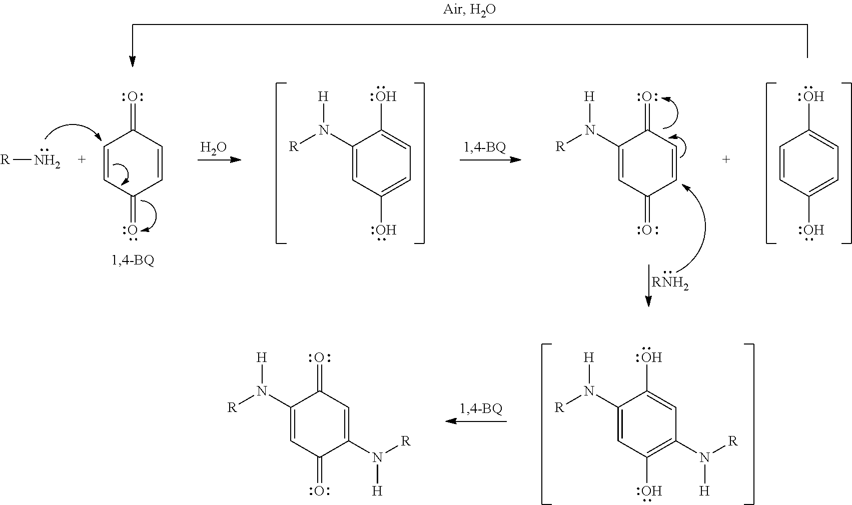

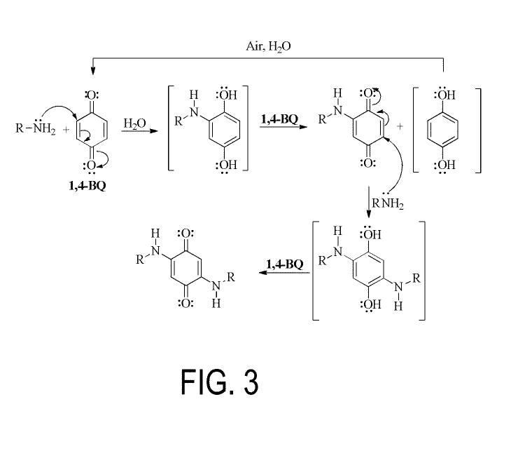

Scheme 1 (Repeated in FIG. 3): One-pot synthesis of benzoquinone derivatives (BQ₁₋₁₄) in 95% methanol. Hydrobenzoquinone is produced as a byproduct.

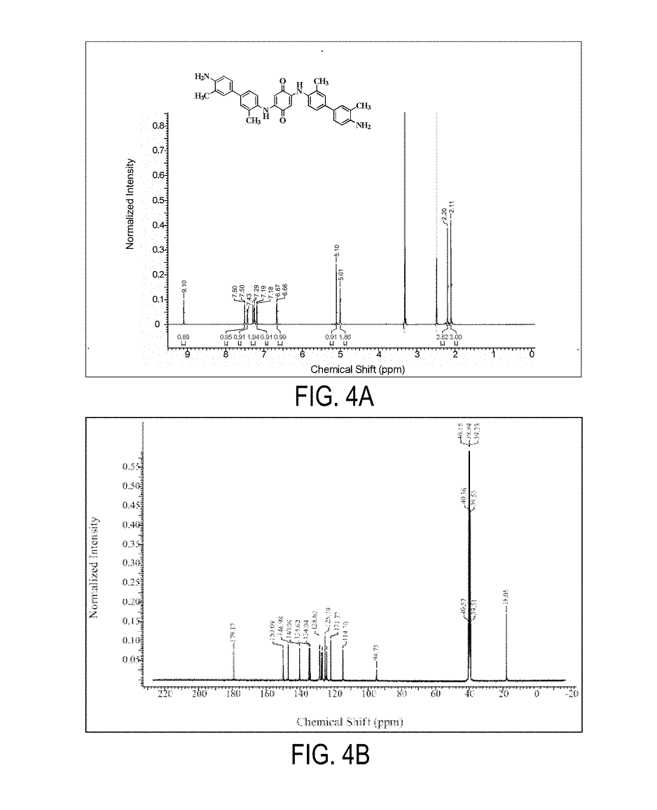

An example is the reaction between 3,3′-dimethyl-(1,1′-biphenyl)-4,4′-diamine and benzoquinone resulted in symmetrically di-substituted benzoquinone (BQ6). Referring also to FIGS. 4(a) and 4(b), ¹H-NMR showed a singlet signal at δ=5.02 ppm equivalent to the two symmetric benzoquinone olefinic protons. The singlet signal at δ−5.09 ppm (exchangeable with D₂O ) is attributed to two amino groups. The signal at δ=9.11 ppm (exchangeable with D2O) is attributed to two identical imino-protons of NH. The four doublets at δ=6.66, 7.18, 7.25 and 7.41 ppm and two singlets at δ=7.28 and 7.50 ppm are attributed to the biphenyl aromatic protons. ¹³C-NMR gave signals at δ18.05 ppm (equivalent to the four methyl carbons) and 14 signals at δ94.75, 114.70, 121.77, 124.06, 125.19, 127.32, 128.22, 128.62, 134.04, 134.62, 140.09, 146.98, 150.09, and 179.17 ppm attributed to the rest 14 aromatic carbons.

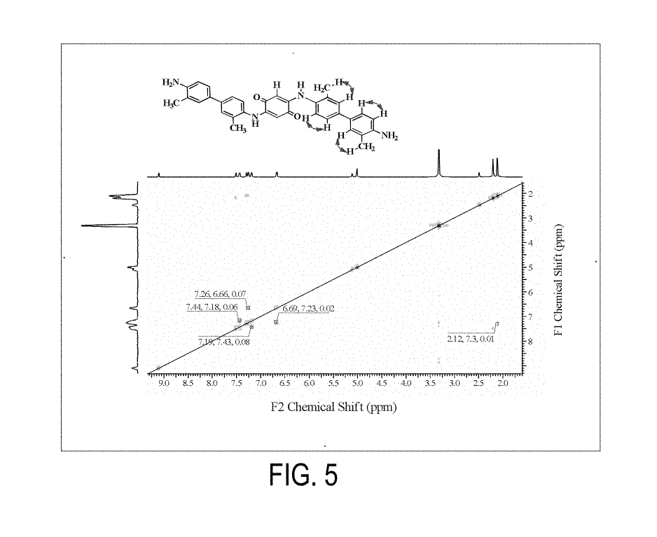

The gCOSY, gHSQC, and gHMBC 2D-NMR spectra gave additional conformation on BQ6 structure. The gCOSY spectra of BQ6 in FIG. 5 shows ¹H-¹H cross peak interactions between the two methyl's protons resonate at δ=2.12 and δ=2.21 ppm and the meta-biphenyl protons resonate at δ=7.28 (1%) and δ=7.50 ppm (1%) respectively. Biphenyl H-2,2′ resonate at δ=6.67 and 7.24 ppm showed cross peaks interactions with H-3,3′ δ=7.28 (5%) and 7.44 ppm (1%), respectively. The amino, imino and BQ-H3 showed no interaction with other protons.

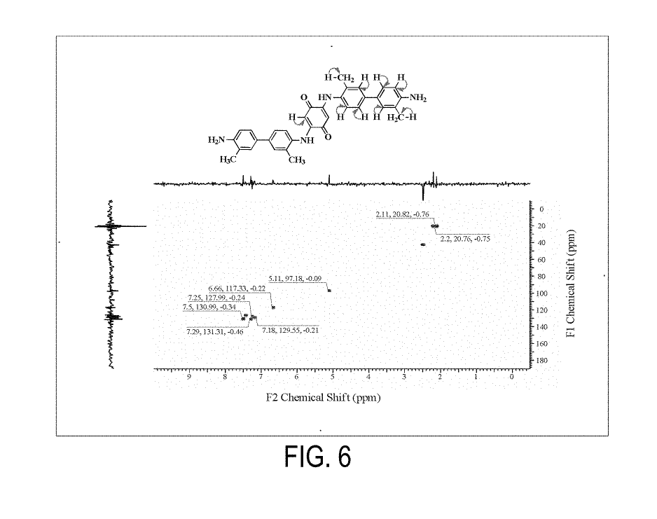

Short-rang HSQC of BQ6 collected in DMSO-d₆ is given in FIG. 6. The methyl protons resonate at δ=2.11 ppm are correlated with the two methyl carbons resonate at δ=20.75 ppm (92%). The singlet BQ protons at δ=5.10 ppm are correlated with BQ C-3 resonate at δ=97.35 ppm (32%). Biphenyl ortho-proton resonates at δ=6.66 ppm is also having a correlation with its carbon resonates at δ=117.49 ppm (25%). Protons resonate at δ=7.25, 7.29 and 7.50 ppm showed correlations with the carbons resonate at δ=127.79 (25%), 131.17 (34%) and 130.95 ppm (46%), respectively.

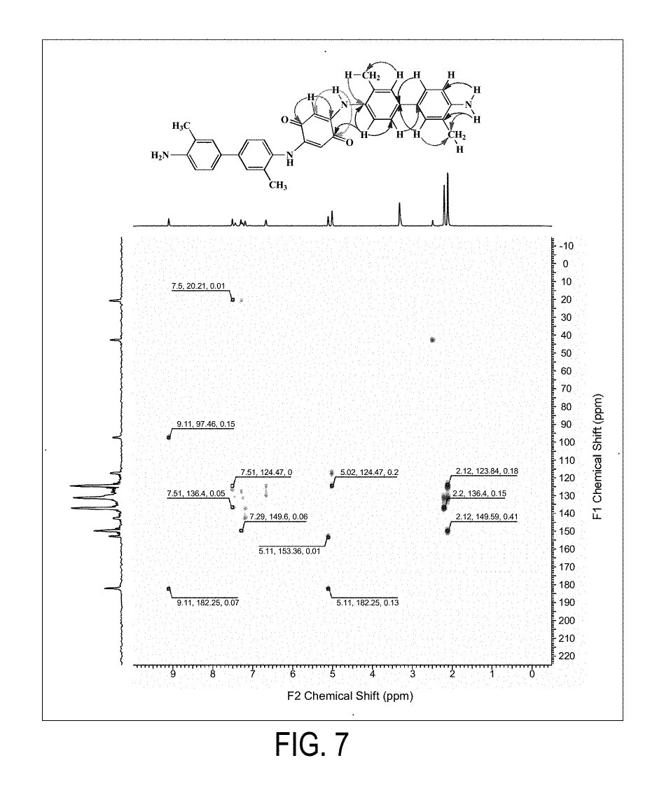

Long-range ¹H-¹³C gHMBC showed a strong correlation between BQ protons resonate at δ=5.11 ppm and the C=O and C-2 resonated at δ=182.14 ppm (2%) at δ=152.22 ppm (3%), respectively (FIGS. 7). The first ortho-methyl protons resonate at δ=2.11 ppm showed strong correlations with the biphenyl carbons resonate at δ=124.06 (44%), 131.10 ppm (48%). The second ortho-methyl protons resonate at δ=2.20 ppm showed strong correlation with the biphenyl carbons resonate at δ=131.10 (34%) and 136.97 ppm (73%). In additions, the amino protons resonated at δ=5.02 ppm are correlated with the biphenyl carbons resonated at δ=117.61 ppm (12%), and δ=124.07 ppm (9%). On the other hand, the imino protons resonated at δ−9.10 ppm are correlated with both BQ C−O resonate at δ=182.14 ppm (11%) and BQ C-3 resonated at δ=97.08 ppm (9%). These results fully confirm the suggested structure for BQ6 and other BQs.

Interaction of obtained BQs with G-quadruplex DNA AGGG(TTAGGG)₃ as examined using UV-Vis, fluorescence, NMR and CD spectroscopy, will be explained in more detail hereinbelow. Binding parameters and melting temperatures will also be investigated hereinbelow. Effects of the synthesized BQ derivatives on human pancreatic cancer cells (L3.6pl and MiaPaCa-2), human lung cancer cells (H1299), human breast cancer cells (MCF-7) and human prostate cancer cells (C42B) cancer cells were investigated in vitro using MTT assay, as will be explained in more detail hereinbelow.

### Chemical Analysis Data for All Synthesized Compounds

Chemical analysis data for all synthesized compounds are described below.

### (BQ1) 2,5-bis-(3-trifluoromethylanilino)-1,4-benzoquinone

Light brown crystals, yield 88% (1.876 g), mp 280-282.5° C.; IR (KBr, ν cm⁻¹): 3257 (N−H), 1641 (C=0); ¹H-NMR (400 MHz, DMSO-d₆) δ ppm 5.86 (2 H, s, ethylene-H-3,6) 7.54-7.72 (8 H, m, (Ar-H)₈) 9.51 (2 H, s, (NH)₂); ¹³C NMR (400 MHz, DMSO-d₆) δ ppm 96.90, 120.69, 122.15, 127.59, 130.98, 139.30, 147.01, 180.60. EI-MS: m/z 292.2, 0.9% (M+), 232.1, 23.1%, 206.1, 61.1%, 149.0, 67.6%, 100, 100%. Calculated CHN analysis for C₂₀H₁₂F₆N₂O_{2:}: C, 56.35%; H, 2.84%; N, 6.57%. Found: C, 55.61%; H, 2.51%; N, 6.31%.

### (BQ2) 2,5-bis-(3,4,5-trifluoroanilino)-1,4-benzoquinone

Brown crystals, yield 92% (1.832 g), mp>350° C.; IR (KBr, ν cm⁻¹): 3227 (N−H), 1642 (C=0); ¹H NMR (400 MHz, DMSO-d₆) δ ppm 5.31 (2 H, s, ethylene-H-3,6) 7.52-7.64 (4 H, m, (Ar-H)₂) 8.93 (2 H, s, (NH)₂); ¹³C NMR (400 MHz, DMSO-d₆) δ ppm 96.90, 120.69, 122.14, 125.63, 127.60, 130.97, 139.23, 146.96, 180.60. EI-MS: m/z 292.2, 0.9% (M+), 232.1, 23.1%, 206.1, 61.1%, 149.0, 67.6%, 100, 100%. Calculated CHN analysis for C₁₈H₈F₆N₂O_{2:} C, 54.28%; H, 2.02%; N, 7.03%. Found: C, 55.64%; H, 2.16%; N, 4.25%.

### (BQ3) 2,5-bis-(4-methoxylanilino)-1,4-benzoquinone

Dark brown crystals, yield 86% (1.507 g), mp 323-325° C.; IR (KBr, ν cm⁻¹): 3457, 3062, 1664 (C=0); ¹H NMR (400 MHz, DMSO-d₆) δ ppm 3.76 (6 H, s, 20CH₃) 5.59 (2 H, s, ethylene-H-3,6) 6.97 (4 H, d, 4Ar-H, J=7.2) 7.26 (4 H, d, 4Ar-H, J=7.2) 9.02 (2 H, s, NH); ¹³C NMR (400 MHz, DMSO-d₆) δ ppm 55.87, 94.94, 115.07, 125.79, 130.95, 148.68, 157,69, 179.68. EI-MS: m/z 292.2, 0.9% (M+), 232.1, 23.1%, 206.1, 61.1%, 149.0, 67.6%, 100, 100%. Calculated CHN analysis for C₂₀H₁₈N₂O₄: C, 68.56%; H, 5.18%; N, 8.00%. Found: C, 69.68%; H, 4.57%; N, 6.92%.

### (BQ4) 2,5-bis-(4-fluoroanilino)-1,4-benzoquinone

Dark brown crystals, yield 83% (1.354 g), mp>350° C.; IR (KBr, νcm⁻¹): 3467, 3228 (N−H), 1640 (C=0); ¹H NMR (400 MHz, DMSO-d₆) δ ppm 5.66 (2 H, s, ethylene-H-3,6) 7.22-7.27 (4 H, 4Ar-H, m) 7.36-7.40 (4 H, 4Ar-H, m) 9.34 (2 H, s, 2NH); ¹³C NMR (400 MHz, DMSO-d₆) δ ppm 95.51, 105.00, 116.38, 116.60, 126.43, 126.51, 134.47, 148.13, 180.09. EI-MS: m/z 292.2, 0.9% (M+), 232.1, 23.1%, 206.1, 61.1%, 149.0, 67.6%, 100, 100%. Calculated CHN analysis for C₁₈H₁₂F₂N₂O₂: C, 66.26%; H, 3.71%; N, 8.59%. Found: C, 67.45%; H, 3.46%; N, 8.54%.

### (BQ5) 2,5-bis-anilino-1,4-benzoquinone

Brown crystals, yield 93% (1.350 g), mp 342-345° C.; IR (KBr, ν cm⁻¹): 3466, 3233 (N−H), 1639 (C=0); ¹H NMR (400 MHz, DMSO-d₆) δ ppm 5.76 (2 H, s, ethylene-H-3,6) 7.32-7.44 (10 H, 10Ar-H, m) 9.33 (2 H, s, 2NH); ¹³C NMR (400 MHz, DMSO-d₆) δ ppm 99.96, 123.71, 129.69, 133.61, 139.27, 144.80, 186.35. EI-MS: m/z 292.2, 0.9% (M+), 232.1, 23.1%, 206.1, 61.1%, 149.0, 67.6%, 100, 100%. Calculated CHN analysis for C₁₈H₁₄N₂O₂: C, 74.47%; H, 4.86%, N, 9.65%. Found: C, 75.05%; H, 4.54%; N, 10.05%.

### (BQ6) 2,5-bis-((3,3′-Dimethyl-1,1′-Biphenyl-4-amine)-4′-amino)-1,4-benzoquinone

Dark brown crystals, yield 78% (2.063 g), mp>350° C.; IR (KBr, νcm⁻¹): 3436, 3225 (N−H), 1633 (C=0); ¹H NMR (400 MHz, DMSO-d₆) δ ppm 2.10 (6 H, s, 2CH3) 2.19 (6 H, s, 2CH₃) 5.02 (4 H, s, 2NH₂) 5.09 (2 H, s, ethylene-H-3,6) 6.65 (2 H, d, 2Ar-H, J=8) 7.17 (2 H, d, 2Ar-H, J=7.6) 7.24 (2 H, d, 2Ar-H, J=8) 7.28 (2 H, s, 2Ar-H) 7.42 (2 H, d, 2Ar-H, J=7.6) 7.50 (2 H, s, 2Ar-H) 9.11 (2 H, s, 2NH); ¹³C NMR (400 MHz, DMSO-d₆) δ ppm 18.05, 94.76, 114.70, 121.78, 124.07, 125.20, 126.83, 127.29, 127.33, 128.23, 128.63, 134.05, 134.63, 140.10, 146.99, 150.10, 179.18. EI-MS: m/z 292.2, 0.9% (M+), 232.1, 23.1%, 206.1, 61.1%, 149.0, 67.6%, 100, 100%. Calculated CHN analysis for C₃₄H₃₂N₄O₂: C, 77.25%; H, 6.10%; N, 10.60%. Found: C, 78.49%; H, 5.95%; N, 11.21%.

### (BQ7) 2,5-bis((2-morpholinoethyl)amino)-1,4-benzoquinone

Brown crystals, yield 55% (1.002 g), mp 184-188° C.; IR (KBr, ν cm⁻¹): 3354, 2980, 2957, 2922, 2869, 2813, 1644 and 1613 (C=0); ¹H NMR (400 MHz, DMSO-d₆) δ ppm 2.46 (8 H, s, 4CH₂) 2.64 (4 H, t, 2CH₂, J=6) 3.20 (4 H, m, 2CH₂) 3.72 (8 H, t, ₄CH₂, J=4.4) 5.28 (2 H, s, ethylene-H-3,6) 7.00 (2 H, s, 2NH); ¹³C NMR (400 MHz, DMSO-d₆) δ ppm 38.55, 53.20, 55.56, 66.85, 93.15, 151.05, 178.31. EI-MS: m/z 292.2, 0.9% (M+), 232.1, 23.1%, 206.1, 61.1%, 149.0, 67.6%, 100, 100%. Calculated CHN analysis for C₁₈H₂₈N₄O₄: C, 59.32%; H, 7.74%; N, 15.37%. Found: C, 58.52%; H, 7.64%; N, 13.69%.

### (BQ8) 2,5-bis-(cyclopentylamino)-1,4-benzoquinone

Brick red crystals, yield 73% (1.001 g), mp 289-291° C.; IR (KBr, ν cm⁻¹): 3467, 3252, 2956, 2867, 1634 (C=0); ¹H NMR (400 MHz, DMSO-d₆) δ ppm 1.48-1.64 (12 H, m, 6CH₂) 1.88 (4 H, m, 2CH₂) 3.72 (2 H, m, 2CH) 5.24 (2 H, s, ethylene-H-3,6) 7.33 (2 H, d, 2NH, J=7.2); ¹³C NMR (400 MHz, DMSO-d₆) δ ppm 24.28, 32.01, 53.76, 93.28, 151.09, 177.77. EI-MS: m/z 292.2, 0.9% (M+), 232.1, 23.1%, 206.1, 61.1%, 149.0, 67.6%, 100, 100%. Calculated CHN analysis for C₁₆H₂₂N₂O₂: C, 70.04%; H, 8.08%; N, 10.21%. Found: C, 72.00%; H, 8.27%; N, 10.16%.

### (BQ9) 2,5-bis((2-(pyrrolidin-l-yl)ethyl)amino)-1,4-benzoquinone

Dark brown crystals, yield 53% (0.881 g), mp 159-162° C.; IR (KBr, ν cm⁻¹): 3465, 1643 and 1621 (C=0); ¹H NMR (400 MHz, DMSO-d₆) δ ppm 1.65 (8 H, s, 4CH₂) 2.43 (8 H, s, 4CH₂) 2.59 (4 H, t, 2CH_{2,} J=6.4) 3.20 (4 H, m, 2CH₂) 5.23 (2 H, s, ethylene-H-3,6) 7.47 (2 H, s, 2NH); ¹³C NMR (400 MHz, DMSO-d₆) δ ppm 23.57, 41.18, 53.33, 53.85, 92.57, 151.53, 177.64. EI-MS: m/z 292.2, 0.9% (M+), 232.1, 23.1%, 206.1, 61.1%, 149.0, 67.6%, 100, 100%. Calculated CHN analysis for C₁₈H₂₈N₄O₂: C, 65.03%; H, 8.49%; N, 16.85%. Found: C, 65.00%; H, 8.25%; N, 16.72%.

### (BQ10) 2,5-bis-(4-trifluoromethylbenzylamino)-1,4-benzoquinone

Brick red crystals, yield 61% (1.386 g), mp 271-274° C.; IR (KBr, ν cm⁻¹ ): 3274, 1643 and 1621 (C=0); ¹H NMR (400 MHz, DMSO-d₆) δ ppm 4.45 (4 H, d, 2benzyl-CH_{2,} J=6.8) 5.16 (2 H, s, ethylene-H-3,6) 7.48 (4 H, d, 4Ar-H, J=8) 7.67 (4 H, d, 4Ar-H, J=8) 8.34 (2 H, t, 2NH, J−6); ¹³C NMR (400 MHz, DMSO-d₆) δ ppm 45.01, 93.96, 125.81, 125.84, 128.31, 142.79, 151.24, 178.37. EI-MS: m/z 292.2, 0.9% (M+), 232.1, 23.1%, 206.1, 61.1%, 149.0, 67.6%, 100, 100%. Calculated CHN analysis for C₂₂H₁₆F₆N₂O₂: C, 58.15%; H, 3.55%; N, 6.17%. Found: C, 57.10%; H, 3.71%; N, 6.16%.

### (BQ11) 2,5-bis-(4-fluorobenzylamino)-1,4-benzoquinone

Brick red crystals, yield 86% (1.524 g), mp 248-250° C.; IR (KBr, ν cm⁻¹): 3281, 1643 and 1606 (C=0); ¹H NMR (400 MHz, DMSO-d₆) δ ppm 4.32 (4 H, d, 2benzyl-CH_{2,} J=6.8) 5.18 (2 H, s, ethylene-H-3,6) 7.12 (4 H, 4Ar-H, m) 7.31 (4 H, 4Ar-H, m) 8.25 (2 H, t, 2NH, J=6.8); ¹³C NMR (400 MHz, DMSO-d₆) δ ppm 44.78, 93.72, 115.56, 115.78, 129.68, 129.77, 133.99, 134.02, 151.22, 160.54, 162.96, 178.28. EI-MS: m/z 292.2, 0.9% (M+), 232.1, 23.1%, 206.1, 61.1%, 149.0, 67.6%, 100, 100%. Calculated CHN analysis for C₂₀H₁₆F₂N₂O₂: C, 67.79%; H, 4.55%; N, 7.91%. Found: C, 70.28%; H, 4.78%; N, 6.46%.

### (BQ12) 2,5-bis-(3,4-dimethoxybenzylamino)-1,4-benzoquinone

Brick red crystals, yield 83% (1.820 g), mp 256.5-258.5° C.; IR (KBr, ν cm⁻¹): 3299 (N−H), 3006, 2934, 2838, 1641 (C=0); ¹H NMR (400 MHz, DMSO-d₆) δ ppm 3.69 (12 H, s, 40CH₃) 4.25 (4 H, d, 2benzyl-CH_{2,} J=6.4) 5.19 (2 H, s, ethylene-H-3,6) 6.76-6.93 (6 H, 6Ar-H, m) 8.15 (2 H, t, 2NH, J=6.4); ¹³C NMR (400 MHz, DMSO-d₆) δ ppm 45.41, 55.90, 55.93, 93.63, 111.82, 112.17, 119.91, 130.07, 148.43, 149.20, 151.32, 178.16. EI-MS: m/z 292.2, 0.9% (M+), 232.1, 23.1%, 206.1, 61.1%, 149.0, 67.6%, 100, 100%. Calculated CHN analysis for C₂₄H₂₆N₂O₆: C, 65.74%; H, 5.98%; N, 6.39%. Found: C, 64.85%; H, 5.77%; N, 6.37%.

### (BQ13) 2,5-bis-(benzylamino)-1,4-benzoquinone

Brown crystals, yield 77% (1.226 g), mp 254.5-256° C.; IR (KBr, ν cm⁻¹): 3467, 3277 (N−H), 1643 (C=0); ¹H NMR (400 MHz, DMSO-d₆) δ ppm 4.34 (4 H, d, 2 benzyl-CH_{2,} J=6.8) 5.16 (2 H, s, ethylene-H-3,6) 7.20-7.33 (10 H, 10Ar-H, m) 8.25 (2 H, t, 2NH, J=6.8); ¹³C NMR (400 MHz, DMSO-d₆) δ ppm 45.56, 93.70, 127.59, 127.63, 128.93, 137.82, 151.38, 178.21. EI-MS: m/z 292.2, 0.9% (M+), 232.1, 23.1%, 206.1, 61.1%, 149.0, 67.6%, 100, 100%. Calculated elemental analysis for C₂₀H₁₈N₂O₂: C, 75.45; H, 5.70; N, 8.80. Found: C, 77.37; H, 5.70; N, 9.61.

### (BQ14) 2,5-bis-(3,5-ditrifluoromethylbenzylamino)-1,4-benzoquinone

Brick red crystals, yield 66% (1.948 g), mp 237-239° C.; IR (KBr, ν cm⁻¹): 3337(N−H), 3062, 2932, 1642 (C=0); ¹H NMR (400 MHz, DMSO-d₆) δ ppm 4.52 (4 H, d, 2benzyl-CH_{2,} J=6.4) 5.34 (2 H, s, ethylene-H-3,6) 7.99 (2 H, s, 2Ar-H) 8.03 (4 H, s, 4Ar-H) 8.40 (2 H, t, 2NH, J=6.4 Hz); ¹³C NMR (400 MHz, DMSO-d₆) δ ppm 44.43, 94.05, 121.54, 122.41, 125.12, 128.78, 130.49, 130.82, 141.61, 151.00, 178.68. EI-MS: m/z 292.2, 0.9% (M+), 232.1, 23.1%, 206.1, 61.1%, 149.0, 67.6%, 100, 100%. Calculated CHN analysis for C₂₄H₁₄F₁₂N₂O₂: C, 48.83%; H, 2.39%; N, 4.75%. Found: C, 50.11%; H, 2.77%; N, 4.71%.

### Drug-DNA Interaction

Circular dichroism is a well-established method for studying changes in DNA conformation. G-quadruplex DNA showed a hybrid (parallel-antiparallel) structure characterized by a negative band at 235 nm and two positive bands at 253 nm and 293 nm. Additions of up to 5% DMSO and 0.1% Tween-80 did not change the hybrid G-quadruplex conformation indicating that they can be safely used for studying DNA interactions with the BQ compounds.

In the followings, Interactions of the BQs with DNA were studied using UV-Vis absorption, fluorescence, fluorescence quenching, circular dichroism and NMR spectroscopic techniques. The results obtained were used to evaluate binding affinities and stabilization effects towards G-quadruplex DNA.

### UV-Vis Absorption Titration

Four absorption bands were identified in UV-Vis spectra of p-benzoquinones. A strong band at 250 nm (ε=20,000) and a weak band at 300 nm (ε=320) ascribed to π-π* transitions. The other two are assigned to the n-n* singlet-singlet and singlet-triplet transitions and shown as very weak at 400-500 (ε=20-30) and 540 nm (ε=0.2), respectively.

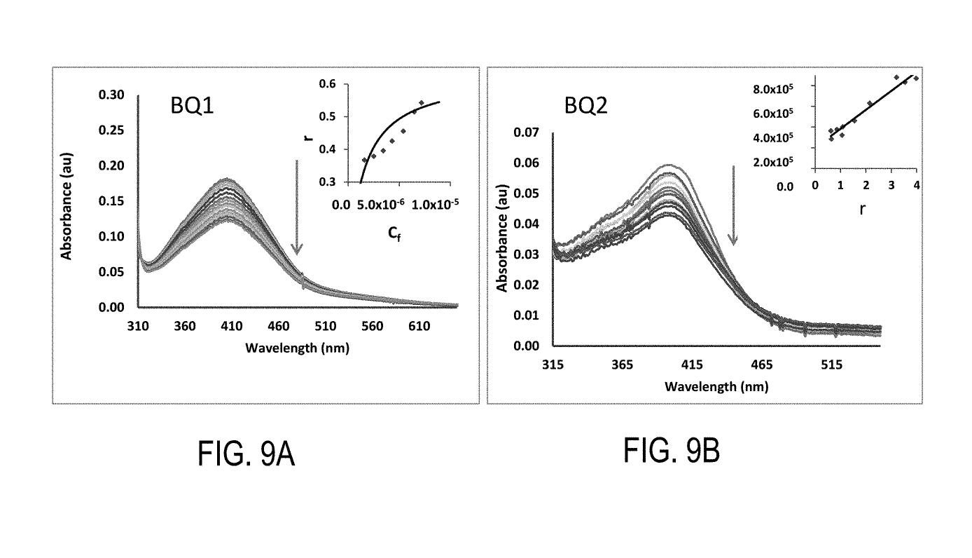

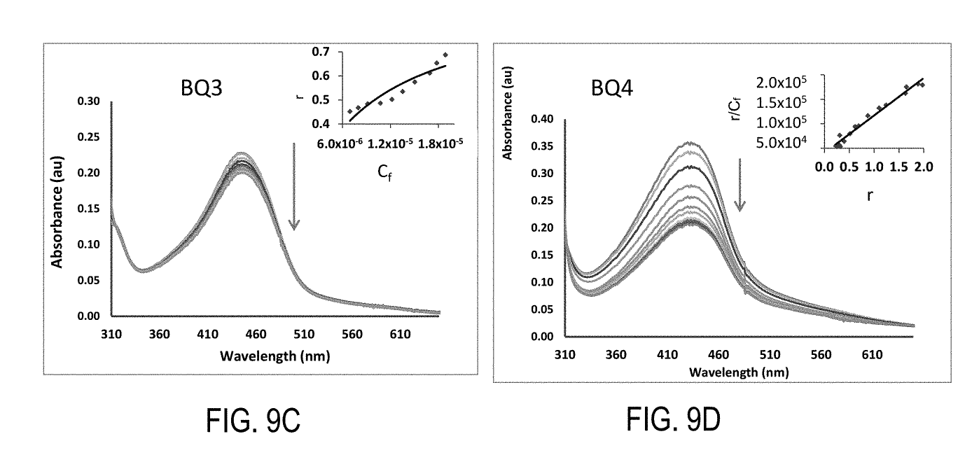

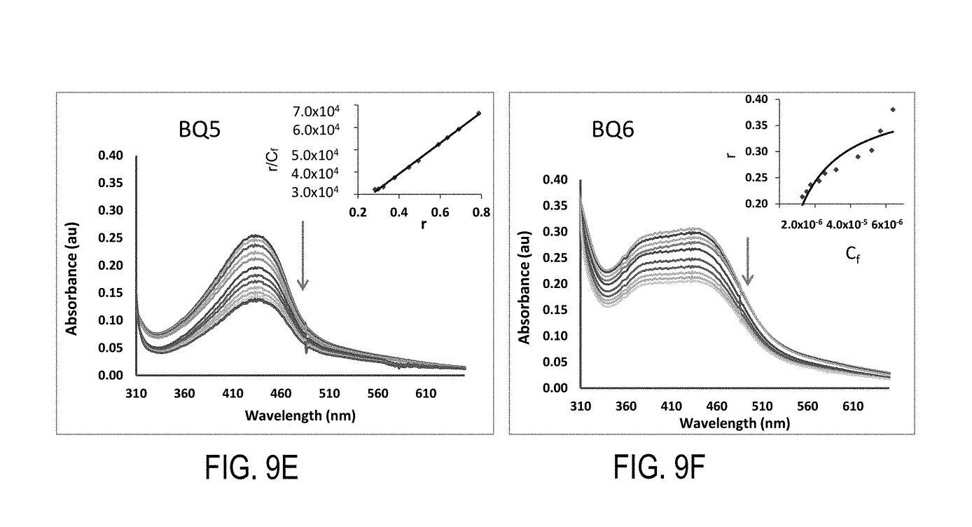

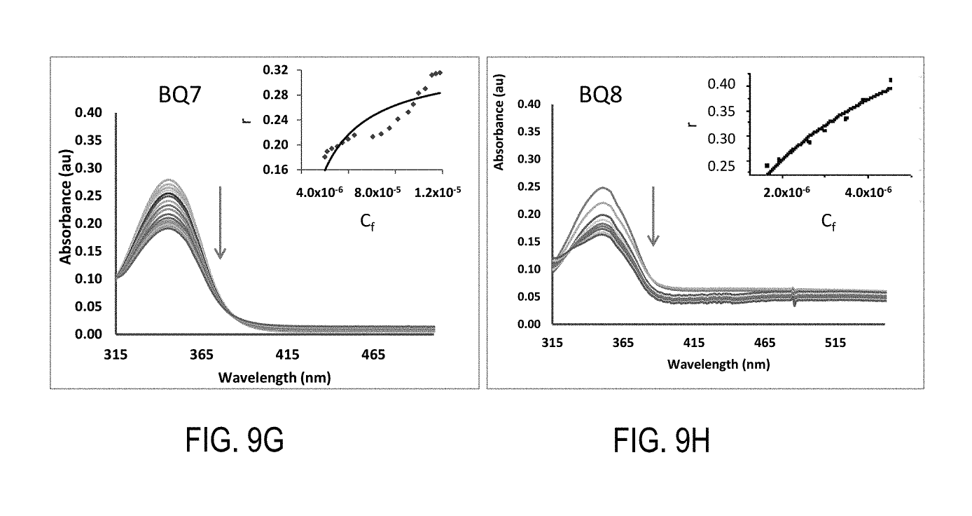

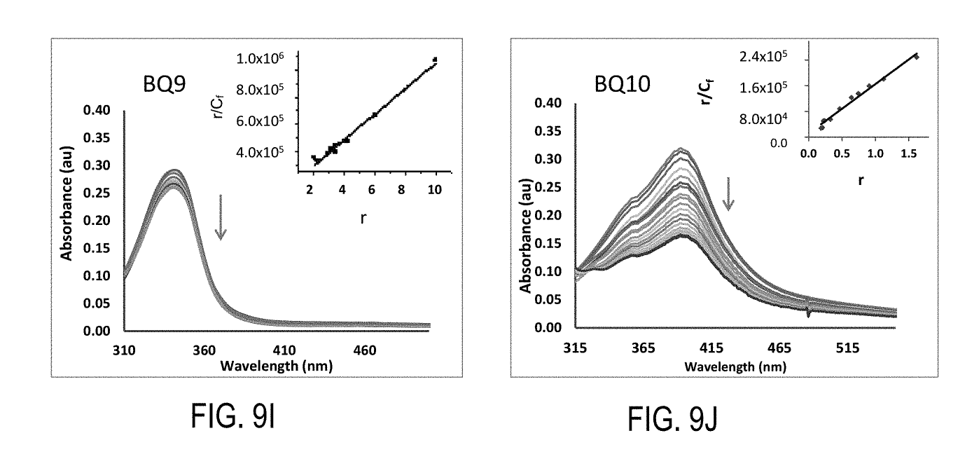

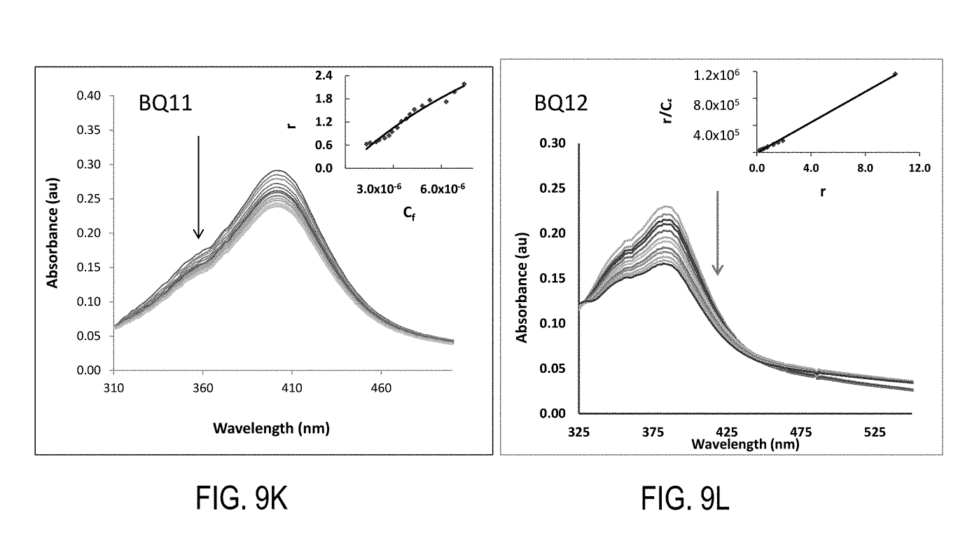

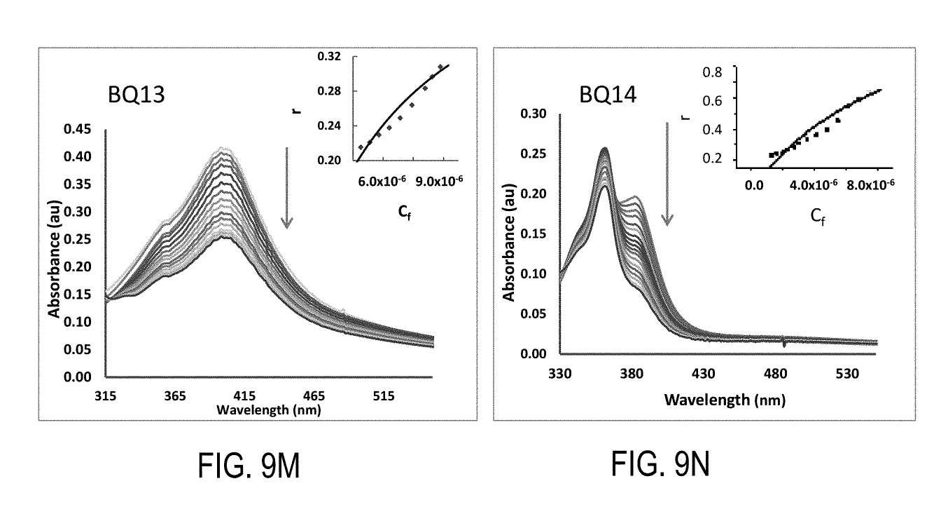

The BQs give absorption bands around 280, 330 and/or460 nm. To avoid interference with DNA absorption bands at 260 and 280 nm, titration of the BQs with DNA was followed at 330 or 460 nm (see FIGS. 9(a) to 9(n)). Additions of G-quadruplex to the BQs resulted in continuous decrease in absorbance (hypochromicity) and slight red shifts. An intercalation binding mode may be suggested for the interaction mechanism of the BQs with G-quadruplex.

### Circular Dichroism Titration

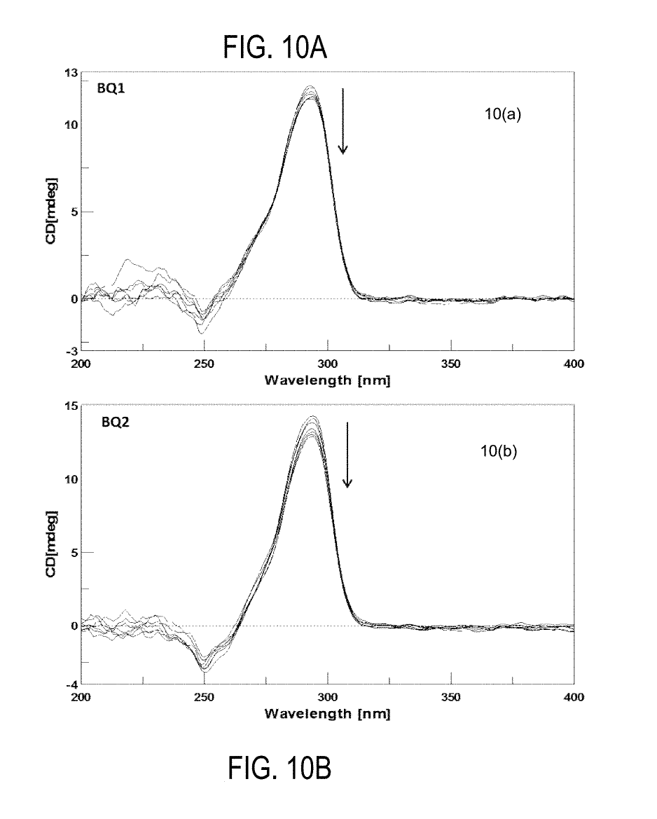

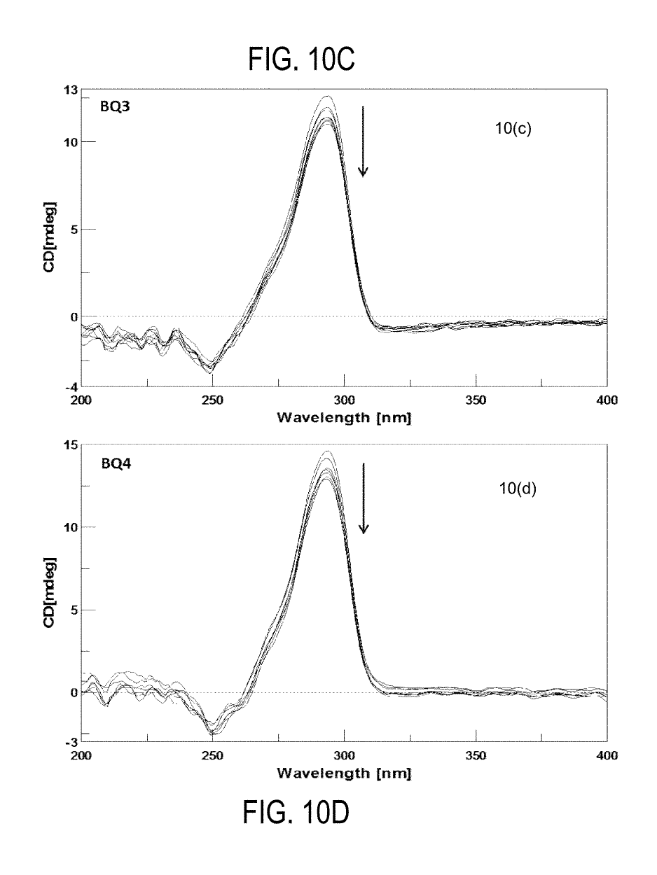

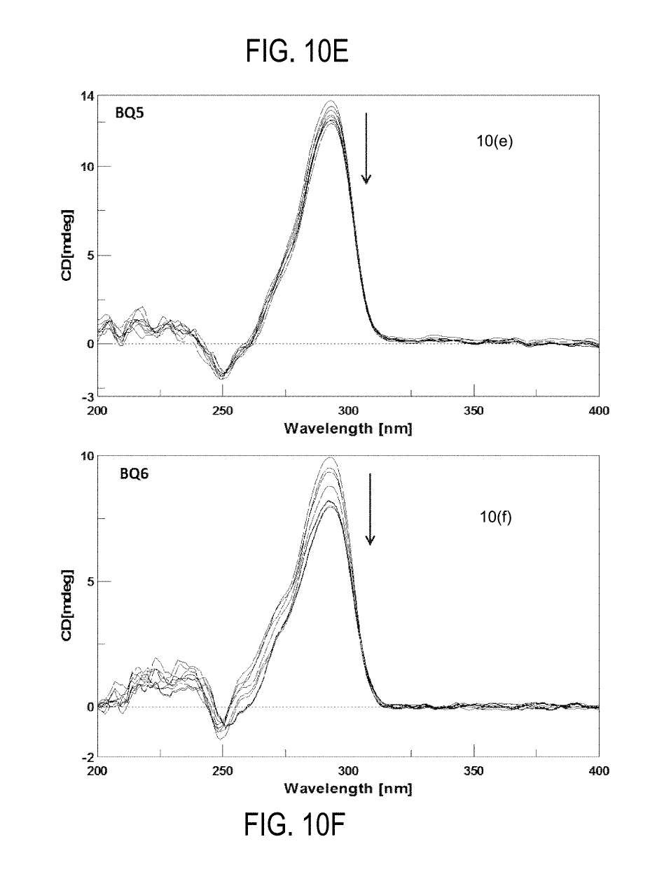

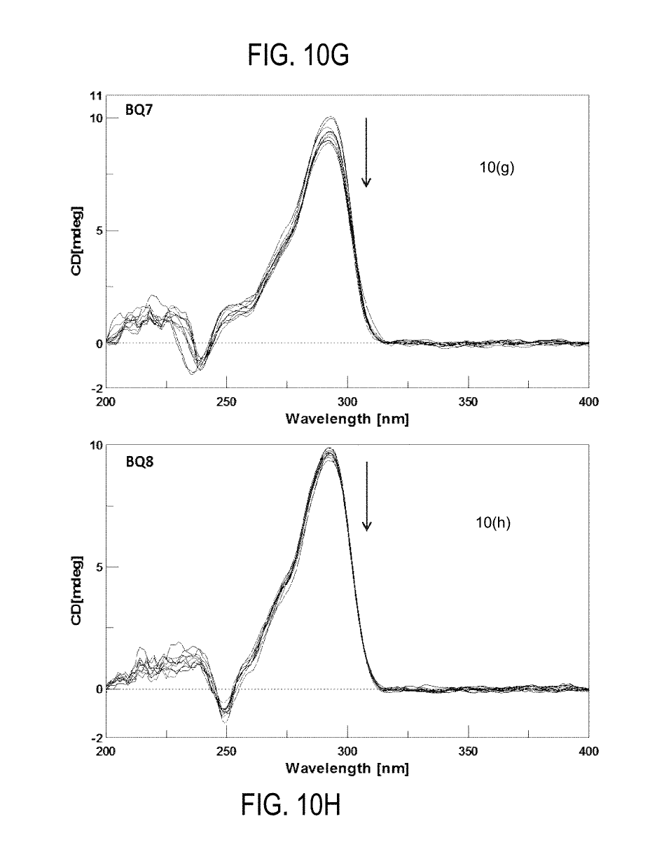

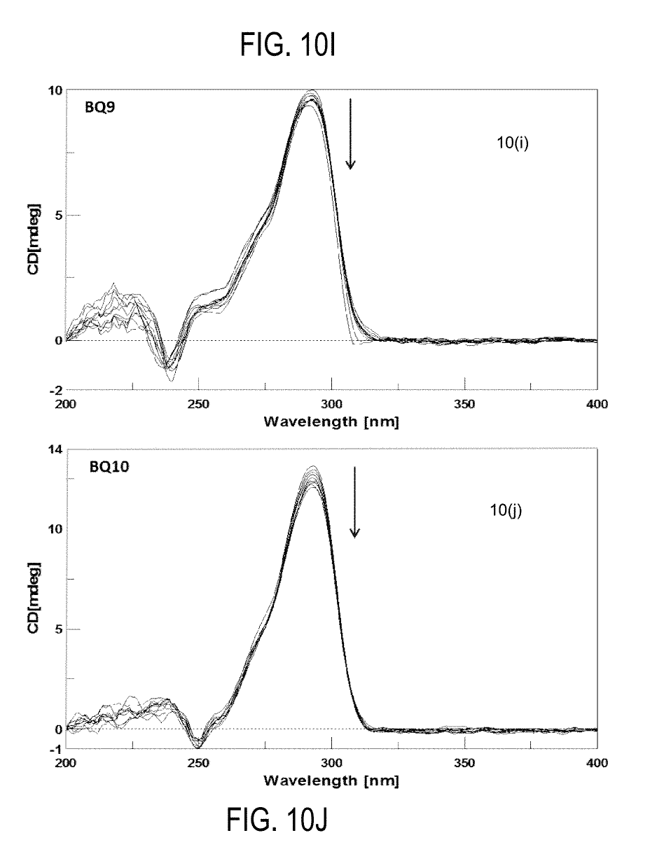

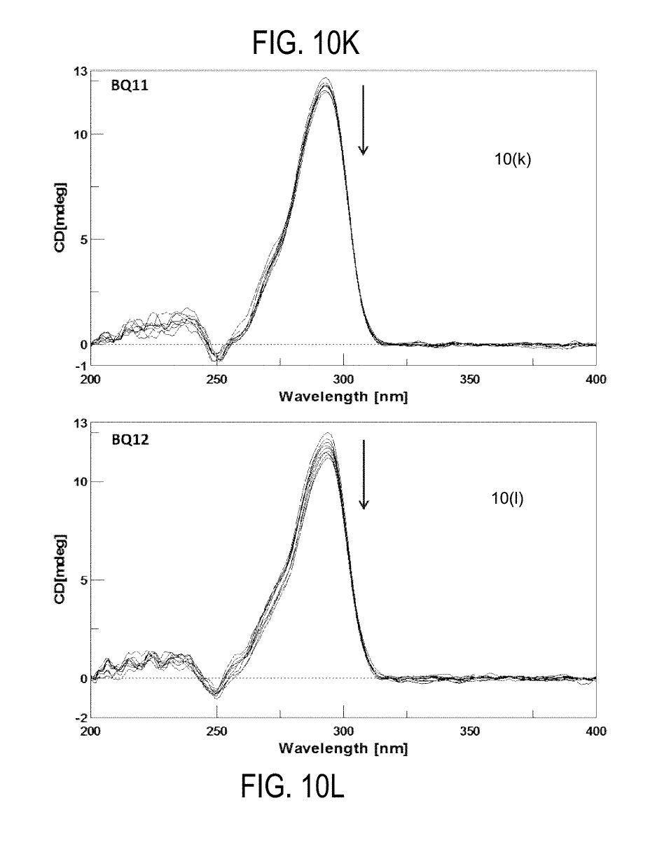

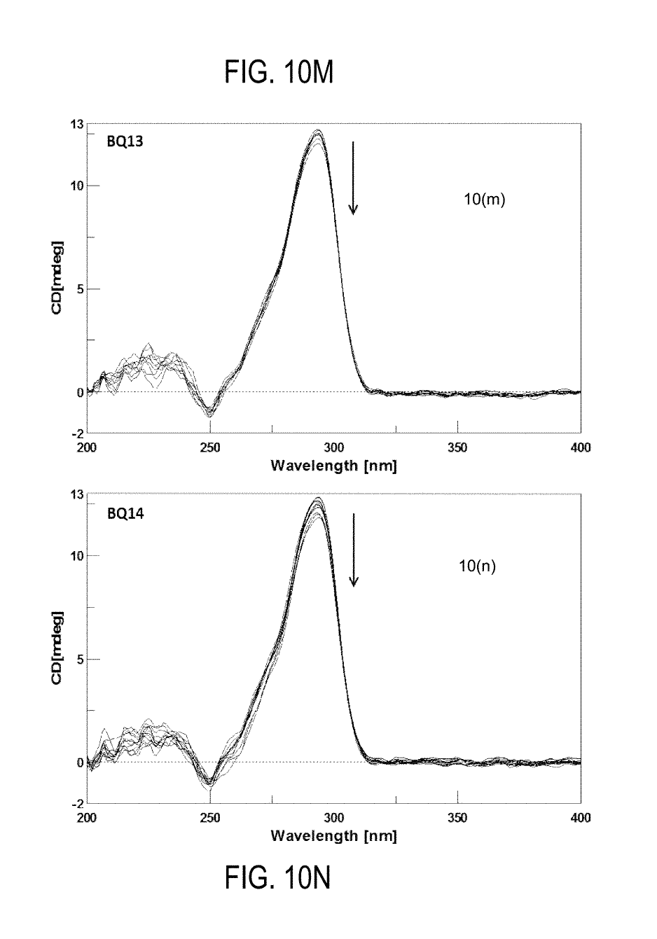

CD titration confirmed the Interactions of BQs with telomere G-quadruplex DNA. In FIGS. 10(a) to 10(n), additions of BQs to G-quadruplex resulted in decrease in CD intensity around 290 nm indicate an intercalation binding mode. No changes in CD bands' positions or shapes indicated that the hybrid conformational structure of G-quadruplex DNA did not change upon interaction.

### Fluorescence Quenching Titration

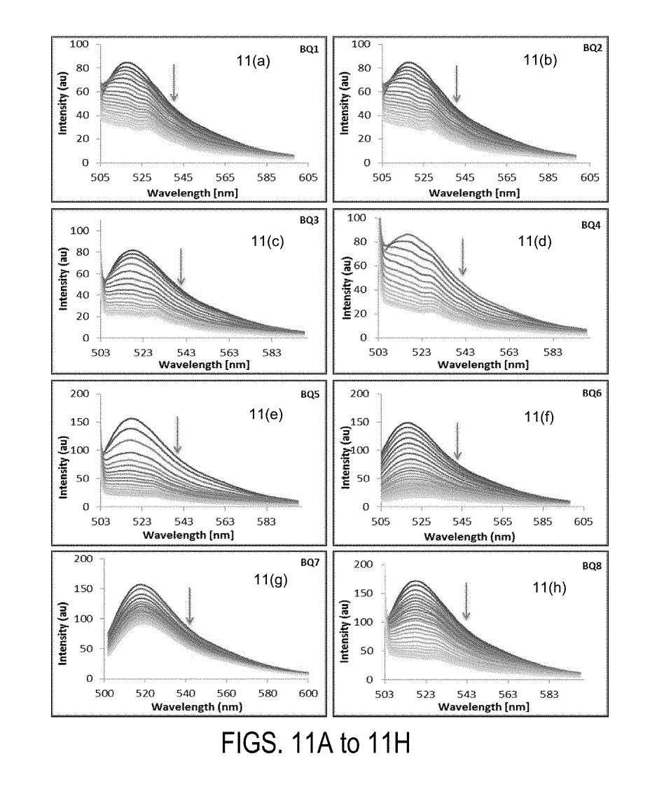

Additional confirmations for the interactions between the BQs with G-quaduplex were obtained from fluorescence quenching titrations. FIGS. 11(a) to 11(n) show quenching of fluorescence intensities of 5′-Fl-G-quaduplex upon additions of the BQs. Since quenching can be attributed to binding in the vicinity of the fluorescent chromophore on 5′-Fl-labbeled G-quadruplex, these results indicate that the BQs bound interacted with the G-quadruplex DNA and confirm the previous results.

Binding Stoichiometry and Binding affinity

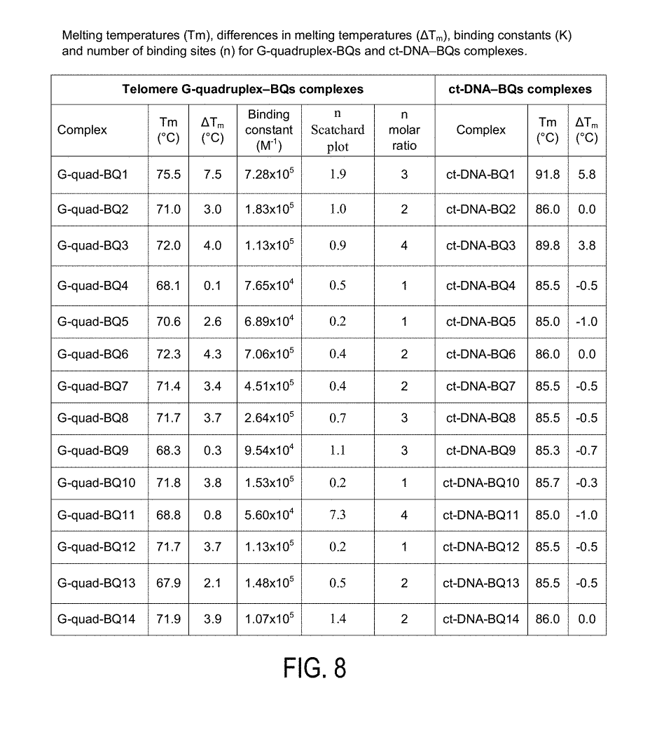

Molar ratio method using UV-Vis titration was used to estimate the binding stoichiometry of the BQs to G-quadruplex DNA. The number of bound BQ molecules per G-quadruplex molecule ranged between 1 and 4 (See Table 2 of FIG. 8).

Scatchard plots based on absorbance measurements gave estimates of the binding constants of BQs towards G-quadruplex DNA. (K). Linear plots indicate one type of equivalent or independent binding sites whereas nonlinear plots indicate more than one type of dependent binding sites that cause neighbor exclusion effect (in which binding on one site may encourage or suppress binding on the another site). Values of binding constants and number of binding sites are given in Table 2 (see FIG. 8). BQ1 has shown the highest binding constant (7.28×10⁵ M⁻¹) while BQ11 has the lowest (5.60×10⁴ M⁻¹). The results indicated that BQ2, BQ4, BQ5, BQ9, BQ10 and BQ12 have one type of binding sites while as the others may have more than one type of binding sites.

### Melting Temperature

Melting temperature gives indications for the stability of DNA-ligand complex and the binding affinity of the ligand towards DNA

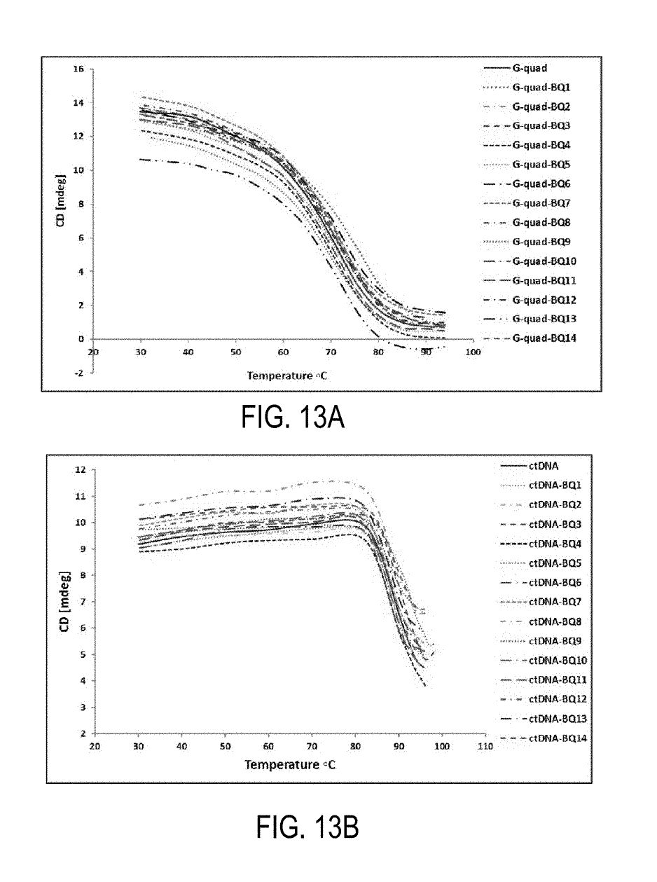

Melting temperatures for G-quadruplex-BQs complexes were estimated using CD spectroscopy (FIG. 13a). Tm values ranged between 68 and 75.5° C. with ΔTm between −0.1 and 7.5° C. were obtained (see FIG. 8, table 2). These values are consistent with their corresponding binding constants. BQ1 has the highest Tm (75.5° C.) and BQ11 has the lowest. BQ1 and BQ6 gave the best stabilizing effect for G-quadruplex DNA (ΔTm=7.5 and 4.3° C., respectively) whereas BQ4 gave the lowest stabilizing effect with (ΔTm=0.1° C.). Other BQs have intermediate stabilizing effects on G-quadruplex DNA (see table 2 of FIG. 8 and see also FIG. 13(a)).

Comparison of melting temperatures of G-quadruplex and duplex DNAs complexes can also give an indication on their selectivity towards both DNAs. Higher ΔTm change gives higher selectivity. FIG. 13b shows the melting temperatures for ct-DNA complexed with the BQs. Tm values ranged in −1.0 and 0.0 with exception of BQ1 and BQ3 gave Tm values of 5.8 and 3.8° C., respectively (see table 2 of FIG. 8). These results indicated that the BQs are highly stabilizing G-quadruplex DNA and destabilizing duplex ct-DNA. The results are also consistent with the corresponding binding constants ranged in 5.6×10⁴−7.28×10⁵ M⁻¹ shown in table 2 of FIG. 8.

### Selectivity

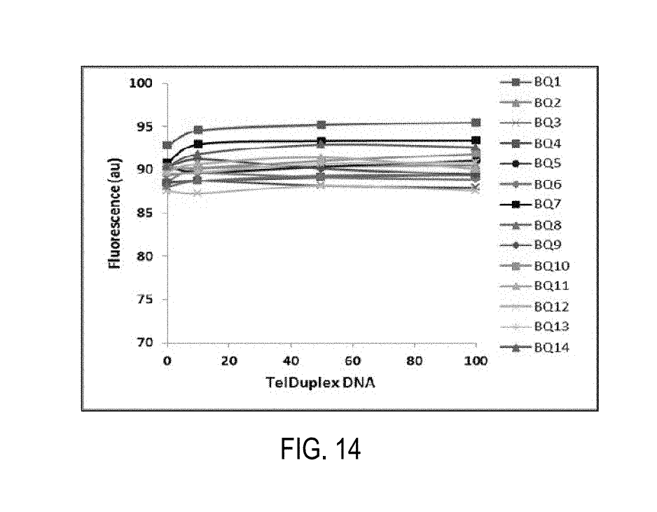

Selectivity of the BQs towards G-quadruplex DNA in the presence of telomere dsDNA was examined. FIG. 14 shows the fluorescence intensity (quenching) of 5′-Flu-TelQ and BQs complexes in the presence of 0, 10, 50 and 100 folds of telomere dsDNA. Additions of telomere dsDNA resulted in slight changes (±2.0%) in fluorescence quantum yields of BQs-Fl-G-quadruplex complexes. These results indicated stable G-quadruplex-BQs complexes with high selectivity towards G-quadruplex DNA over duplex.

Based on the differences between ΔTm (G-quadruplex) and ΔTm (ct-DNA) given in table 2 of FIG. 8, one can assume that the BQs stabilize G-quadruplex DNA by several folds over ct-DNA (0.15-4.3). BQ5, BQ6, BQ7, BQ8, BQ10, BQ12 and BQ14 showed more than 3.5 folds higher selectivity towards G-quadruplex, over ct-DNA. Compounds BQ1, BQ9, BQ11 and BQ13 showed moderate selectivity ranged in 1.0-3.0 folds over ct-DNA. The least selectivity was shown by BQ4 with 0.6 folds while BQ3 gave low selectivity because it stabilized both G-quadruplex and ct-DNA˜ΔTm=0.2° C. (see table 2 of FIG. 8).

Thus, the results from selectivity test using fluorescent G-quadruplex DNA and melting temperatures are consistent and indicated that the BQs have shown higher selectivity towards G-quadruplex over duplex DNA. This conclusion is also supported by the binding constants listed in table 2 of FIG. 8.

### In-Vitro Antitumor Effects

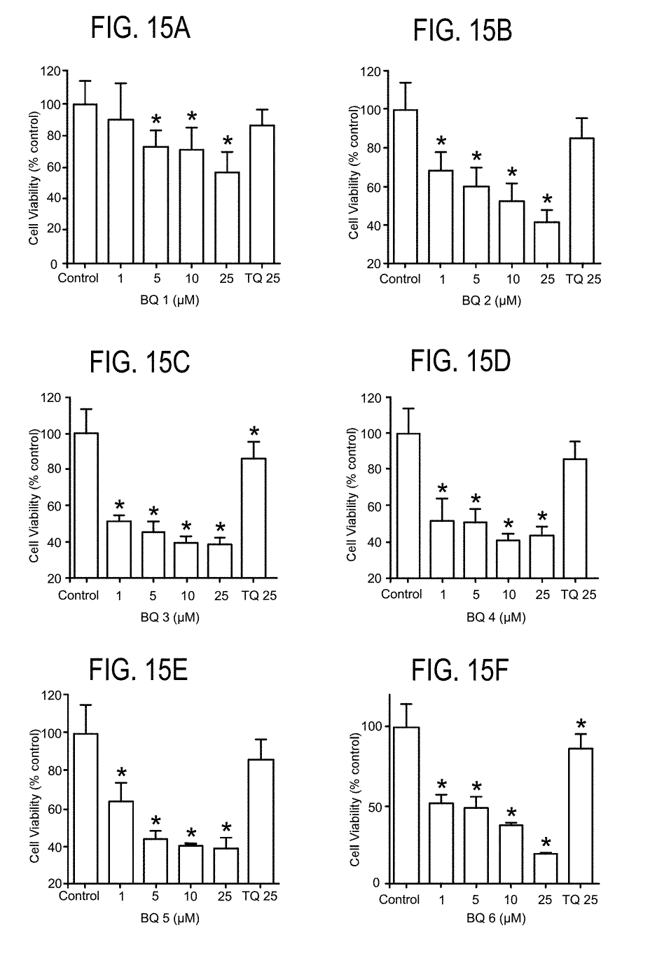

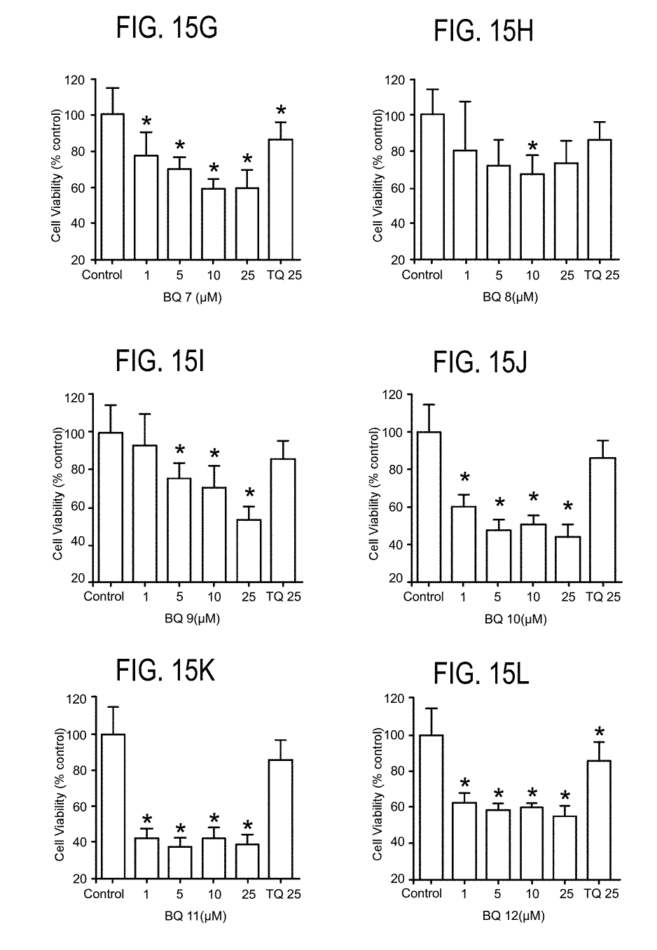

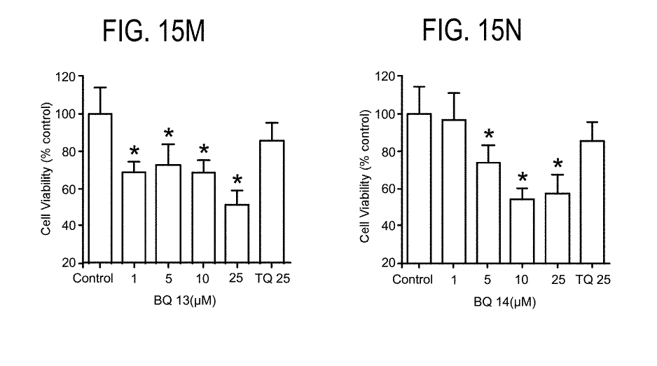

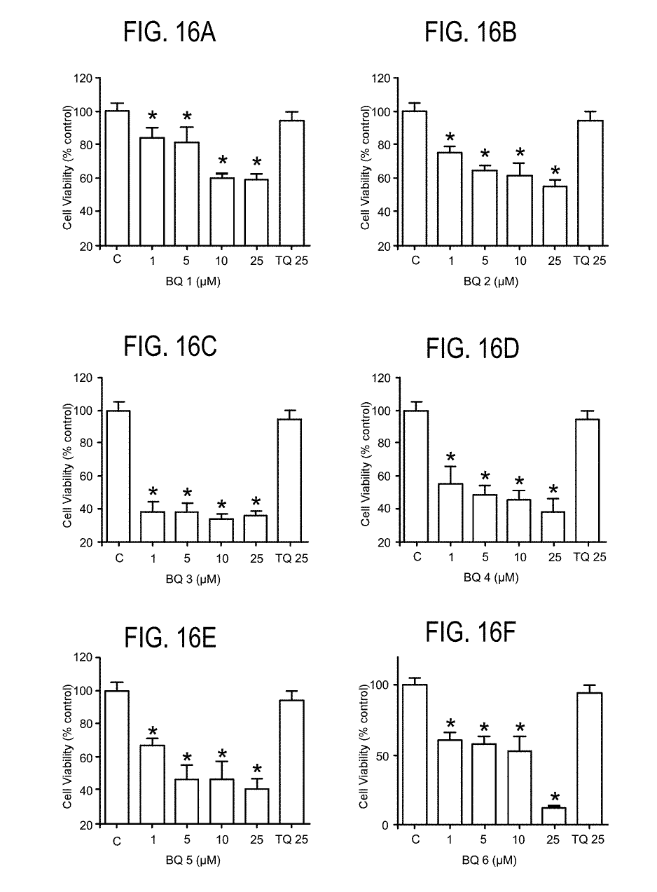

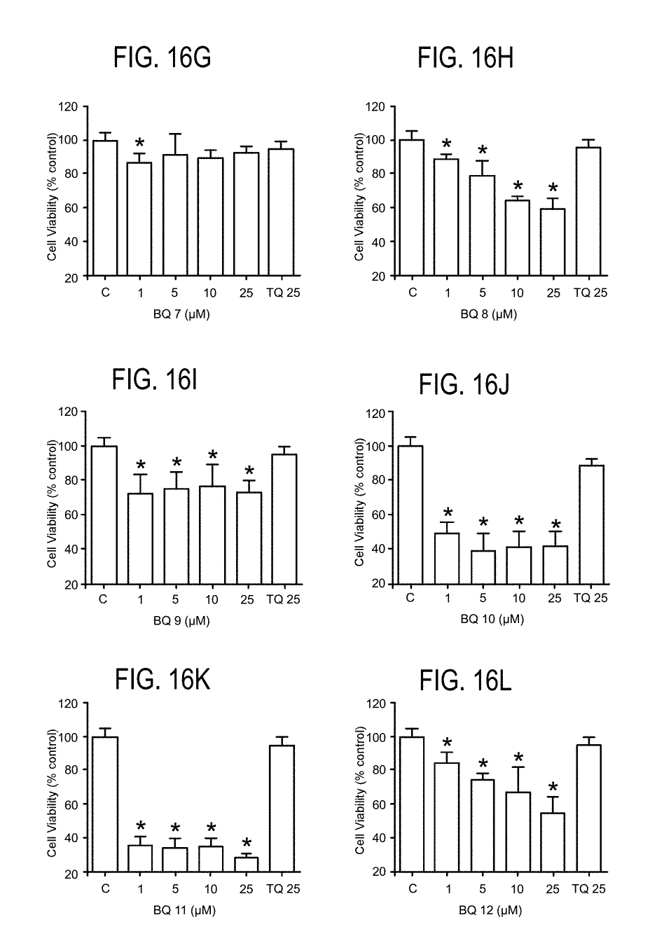

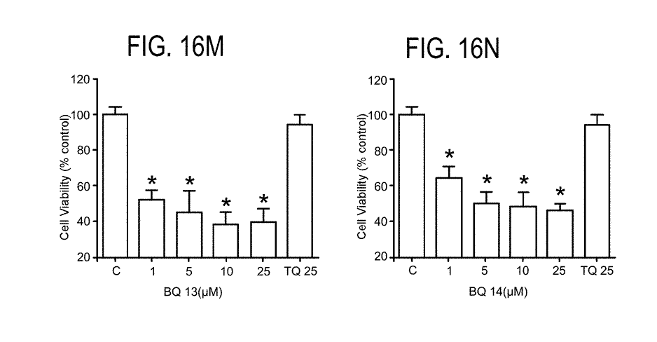

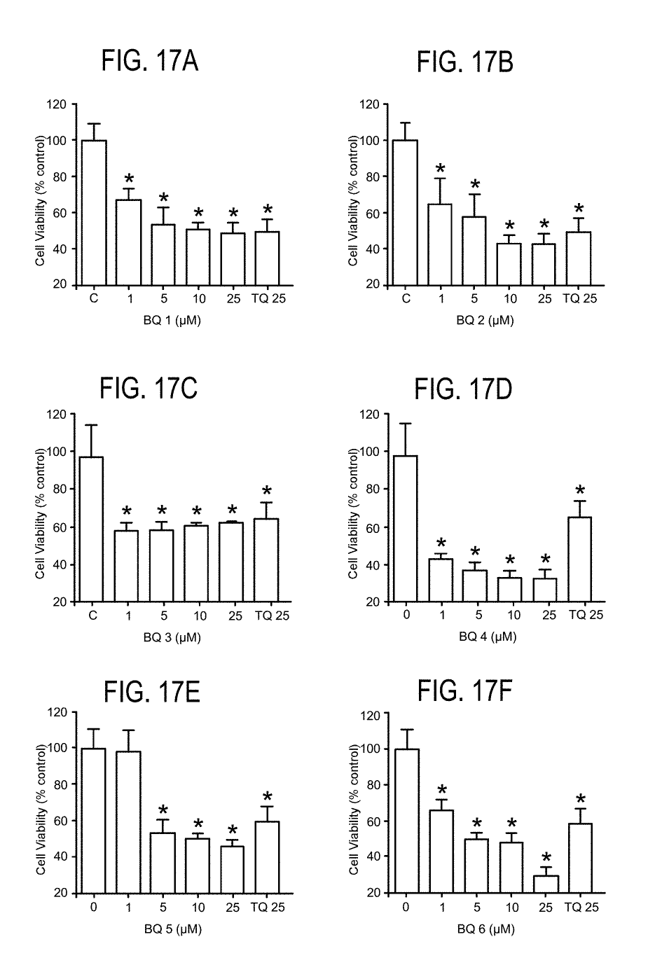

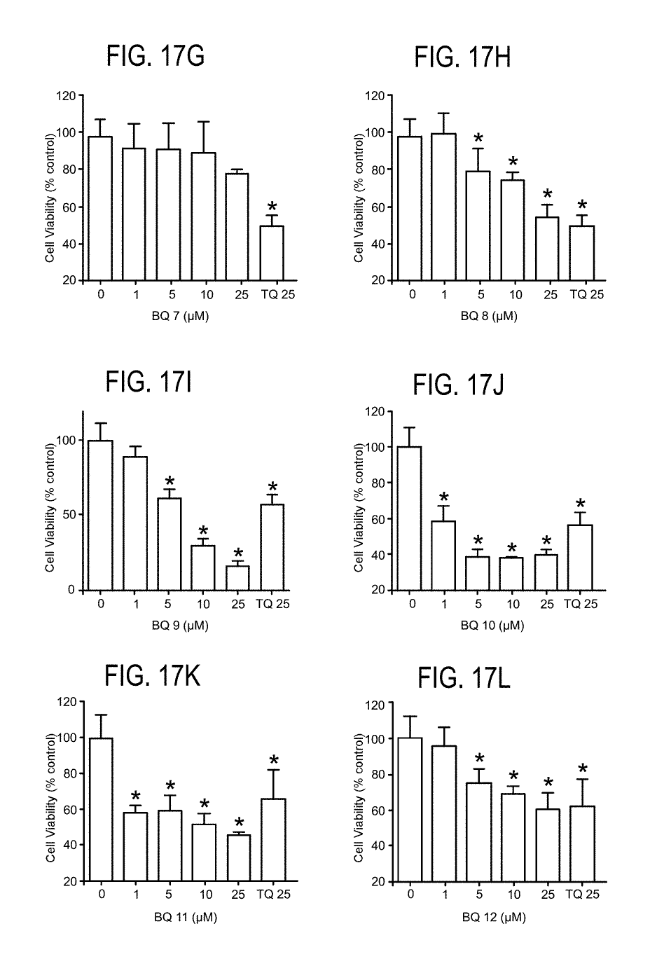

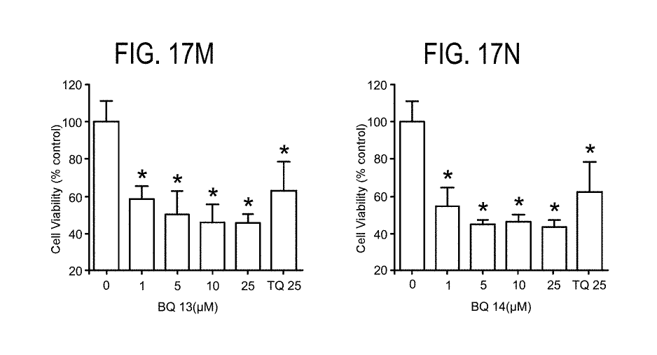

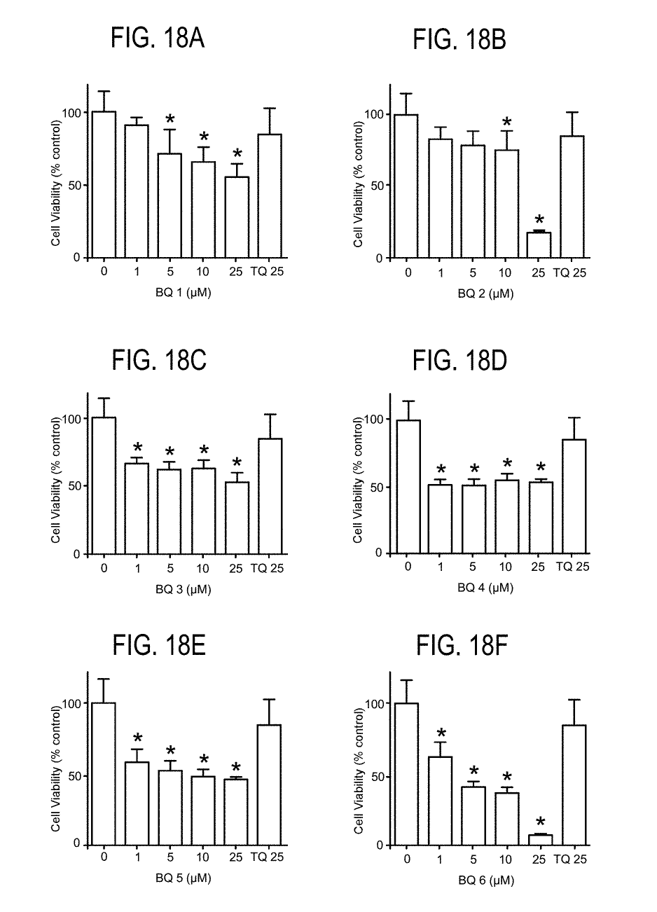

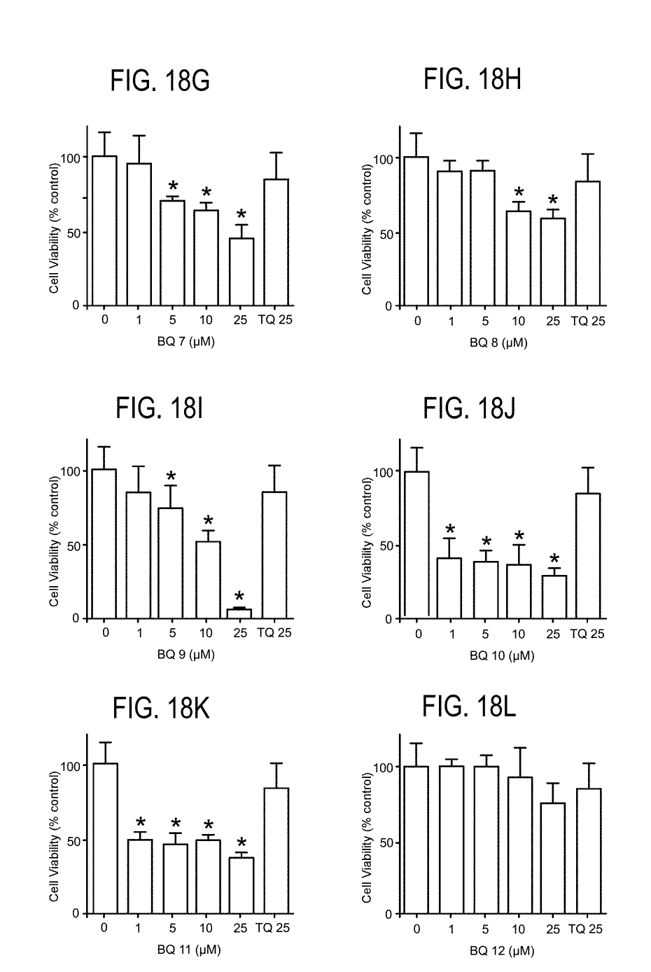

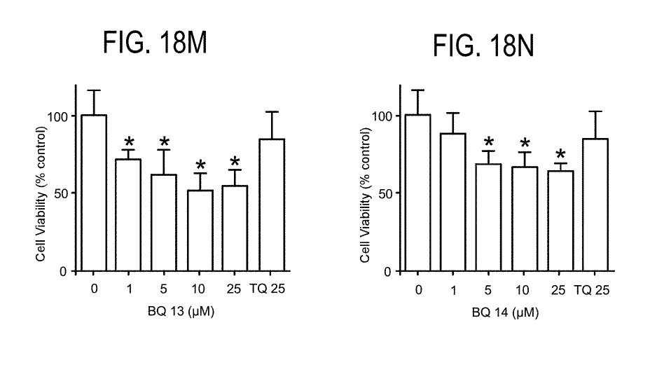

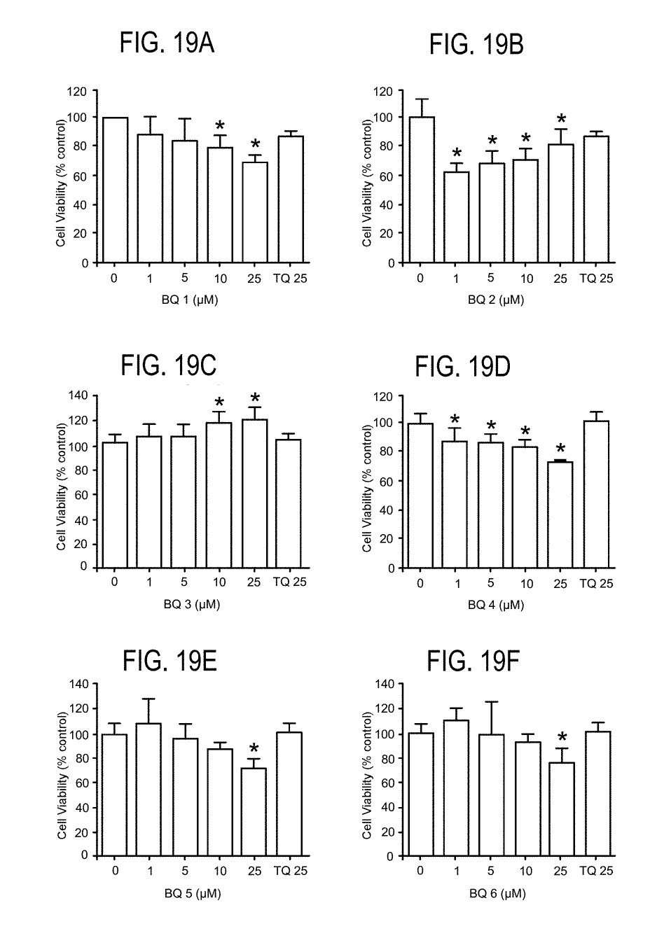

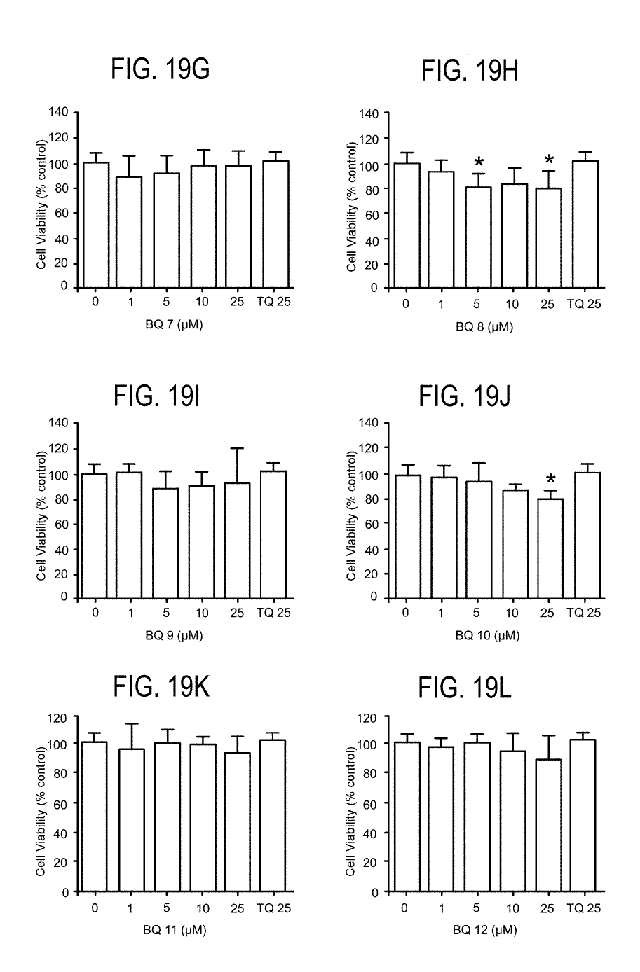

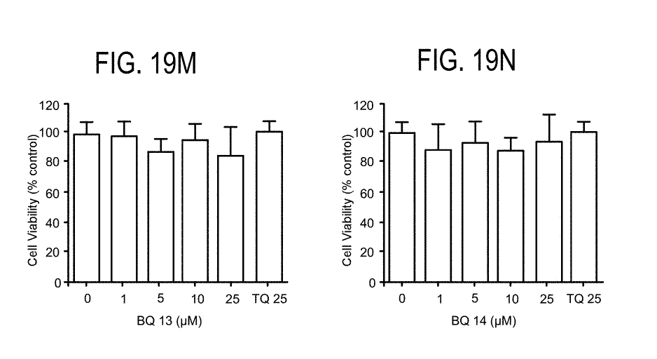

Referring to FIGS. 15 to 19, the BQ compounds were examined against seven cancer cell lines included two pancreatic cancer cell lines (L3.6pl and MiaPaCa-2), one breast cancer cell line (MCF-7), one lung cancer cell line (H1299), one prostate cancer cell line (C42B), one colon cancer cell line (HCT-116) and one non-Hodgkin's lymphoma cancer cell line (WSU-FSCCL). The difference between experimental and control values were assessed by one-way ANOVA followed by Dunnett post hoc multiple comparison test. P<0.05 indicates a significant difference. Thymoquinone was used as reference

The BQs concentrations (1.0-25.0 μM) caused concentration- and time-dependent inhibition in cellular viability of L3.6pl, MiaPaCa-2, H1299, MCF-7 and C42B cells over 72 hours. Colon and lymphoma cancer cells were found insensitive. FIGS. 15-19 show that the BQs are generally more effective than parental thymoquinone. The BQs have shown 50% loss of cell viability in all examined cell lines except human prostate cancer cells (C42B). BQ9 gave the highest loss of cell viability (92.50%) while BQ12 was the lowest (24.16%) in MCF-7. BQ6 gave the highest loss in cell viability (88.15%) relative to the control (DMSO) and BQ7 gave the lowest (7.53%) in MiaPaCa-2. BQ9 gave the highest inhibition in cell viability (84.40%) while BQ7 gave the lowest (20.81%) in H1299. BQ6 gave the highest inhibition in cell viability (80.14%) while BQ8 gave the lowest (26.97%) in L3.6pl, BQ5 gave the highest loss of cell viability (27.25%) and BQ7 gave the lowest (1.29%) in C42B.

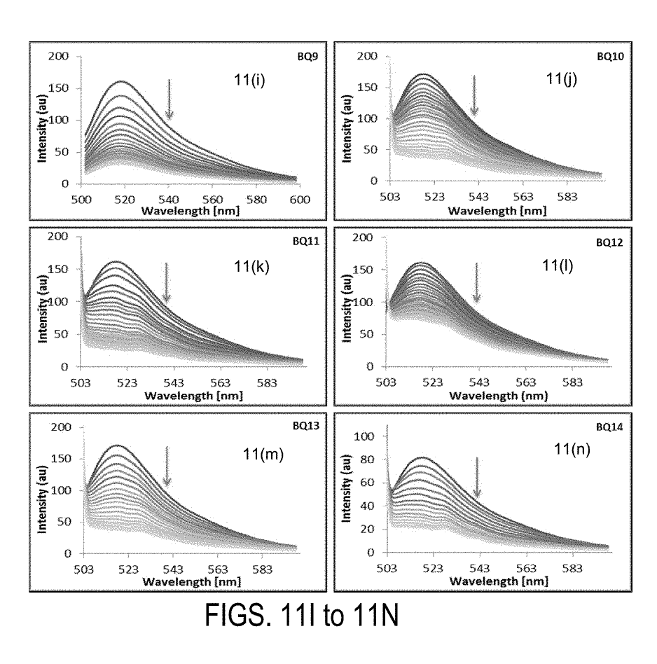

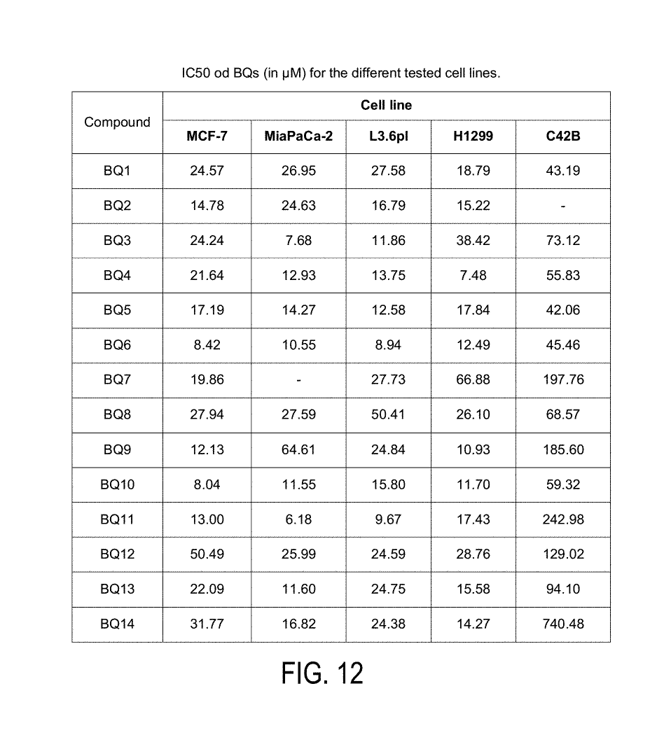

The IC₅₀ concentrations lower than 10 μM are highly desirable in drug syntheses. Referring to FIG. 12, table 3 shows IC₅₀ of the BQs for the examined cell lines at 72 hrs. BQ10 and BQ6 have 8.04 and 8.42 μM for MCF-7. BQ11 and BQ3 have 6.18 and 7.68 μM respectively for MiaPaCa-2. BQ6 and BQ11 have 8.94 and 9.67 μM respectively for L3.6pl. BQ4 have 7.48 μM for H1299 while none of the BQ derivative has IC₅₀ less than 10 μM for C42B cells. H1299 cells seem to be the most sensitive cells to the cytotoxic effects of benzoquinone derivatives (IC₅₀ less than 20 μM) while C42B cells are the least sensitive cells (IC₅₀ higher than 20 μM). As examples, BQ5=42.06, BQ1=43.19 and BQ6=45.46 μM.

The high potency of the BQ derivatives against the tested cell lines is consistent with their binding constants (7.28×10⁵-5.6×10⁴ M⁻¹) and melting temperature values with G-quadruplex DNA. Thus, the anticancer activity of BQs may be attributed to their ability to inhibit telomerase enzyme through stabilizing G-quadruplex DNA. Other mechanisms may also be effective.

### CONCLUSION

The synthesized benzoquinone analogues showed anticancer activity against all examined cancer cell lines except prostate cancer cells (C42B). IC₅₀ ranged between 5.0 and 30.0 μM were obtained in almost all compounds. Compounds BQ6, BQ9, BQ10 and BQ11 have shown the highest potency with IC₅₀ less than 10.0 μM.

Interactions of obtained benzoquinone compounds with telomeric G-quadruplex DNA sequence; AGGG(TTAGGG)_{3;} were tested. The compounds gave affinities ranged in 5.6×10⁴ -7.3×10⁵ M⁻¹ towards G-quadruplex. Selectivity test indicated high selectivity towards G-quadruplex over ct-DNA. Melting temperatures indicated that BQ5, BQ6, BQ7, BQ8, BQ10, BQ12 and BQ14 have ≧3.5 folds higher selectivity towards G-quadruplex over ct-DNA. Compounds BQ1, BQ9, BQ11 and BQ13 showed moderate selectivity (1.0-3.0 folds) while BQ4 gave the least selectivity (0.6 folds).

The BQ derivatives have shown IC50s ranged in 8.04-50.49 for MCF-7, 6.18-64.61 for MiaPaCa-2, 8.94-50.41 for L3.6pl, 7.48-66.88 for H1299 and 42.06-740.48 μM. Human prostate cancer cells (C42B) gave the highest IC50s indicating low potency for tested BQs. BQ9 gave the highest loss of cell viability (92.50%) in MCF-7, BQ6 have shown the highest loss of cell viability (88.15%) in MiaPaCa-2, BQ9 have shown the highest loss of cell viability (84.40%) in H1299, BQ6 have shown the highest loss of cell viability (80.14%) in L3.6pl and BQ5 have shown the highest loss of cell viability (27.25%) in C42B. All the BQ derivatives at 25 μM gave showed higher potency and efficacy than the parental thymoquinone compound except BQ7 and BQ8 in H1299 and BQ3 in C42B.

IC50s of 8.04 and 8.42 μM were obtained for BQ10 and BQ6 in MCF-7, of 6.18 and 7.68 μM for BQ11 and BQ3 in MiaPaCa-2, of 8.94 and 9.67 μM for BQ6 and BQ11 in L3.6pl and of 7.48 μM for BQ4 in H1299. The results also showed that H1299 cells seem to be the highest sensitive cells to the cytotoxic effects of BQs whereas C42B cells are the least sensitive cells. These results are very important since IC50s less than 10.0 μM are recommended for successful development of new drugs

Interaction of synthesized BQ derivatives with G-quadruplex DNA (AGGG(TTAGGG)3) was also investigated using UV-Vis spectrophotometry, fluorescence spectrophotometry, NMR, melting temperature and CD spectroscopy. Binding parameters include binding constant, binding mode; melting temperature; selectivity and binding stoichiometry were evaluated.

The results indicated that the BQs interact with G-quadruplex DNA. Scatchard plots revealed linear and nonlinear correlations indicating binding constants in the range 7.28×10⁵ -5.60×10⁴ M⁻¹ and one or two types of binding sites. BQ1 showed the highest binding constant (7.28×10⁵ M⁻¹) while BQ11 showed the lowest binding constant (1.40×10⁵ M⁻¹).

Similar findings were obtained using melting temperature curves. The BQs have shown to stabilize G-quadruplex. BQ1 gave the highest Tm and ΔTm (75.5 and 7.50° C.) while BQ4 gave the least (68.1 and 0.1° C.). With the exception of BQ1 and BQ3, all other BQs complexes with ct-DNA gave ΔTm≦0.0 indicating that The BQs are destabilizing the duplex ct-DNA. Selectivity test indicated high selectivity towards G-quadruplex over ct-DNA. BQ5, BQ6, BQ7, BQ8, BQ10, BQ12 and BQ14 showed ≧3.5 folds higher selectivity towards G-quadruplex, over ct-DNA Compounds BQ1, BQ9, BQ11 and BQ13 showed moderate selectivity (1.0-3.0 folds) while BQ4 gave the least selectivity (0.15 folds).

As stabilizing G-quadruplex structures in human telomere is expected to inhibit telomerase enzyme found active in almost all cancer cells, the BQ compounds could be good candidates for treating cancers. In additions, they have shown very good potency against tested cancer cell lines

These results revealed a number of novel benzoquinone based compounds with high antitumor efficacy that can be further processed as anticancer agents. More specifically the Benzoquinone derivatives can be used in the manufacture of a pharmaceutical composition for treating cancer.

Most of the BQ derivatives have shown higher binding affinity than parental compound and better selectivity towards G-quadruplex over ct-DNA. The results suggest that these compounds target telomeric G-quadruplex DNA. However, stabilizing DNA may not be the only mechanism by which these compounds act on cancer cells.

While the present invention has been described with respect to specific examples, it should be appreciated that the present invention is not limited to these examples. It is to be believed that one skilled in art, using the preceding description, can utilize the present invention to its fullest extent, and many variations and modifications may present themselves to those of skill in the art without diverting from the scope of the present invention.

1. Compounds of formula (I): 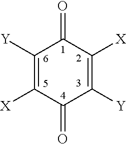
or pharmaceutically acceptable salts or solvates thereof wherein:
one of X and Y is hydrogen and the other one of X and Y is selected from the group consisting of flouroaryl amines, biphenyl amines, amino-pyrrolidines and methoxyphenyl amines.

2. The compounds of formula (I) as claimed in claim 1, or the pharmaceutically acceptable salts or solvates thereof, wherein said other one of X and Y is selected from the group consisting of 3-Trifluoro-methylaniline; 3,4,5-trifluoroaniline; 4-methoxylaniline; 4-fluoroaniline; 3,3′-Dimethyl-1,1′-Biphenyl-4,4′-diamine; 2-(pyrrolidin-1-yl)ethyl)amine; 4-trifluoromethyl-benzylamine ; 4-fluorobenzyl-amine ; 3,4-dimethoxybenzylamine and 3,5-ditrifluoromethyl-benzylamine.

3. The compounds of formula (I) as claimed in claim 1, or the pharmaceutically acceptable salts or solvates thereof, wherein the compounds of formula (I) are selected from the group consisting of:
2,5-bis-(3-trifluoromethylanilino)-1,4-benzoquinone (BQ1);
2,5-bis-(3,4,5-trifluoroanilino)-1,4-benzoquinone (BQ2);
2,5-bis-(4-methoxylanilino)-1,4-benzoquinone (BQ3);
2,5-bis-(4-fluoroanilino)-1,4-benzoquinone (BQ4);
2,5-bis-((3,3′-Dimethyl-1,1′-Biphenyl-4-amine)-4′-amino)-1,4-benzoquinone (BQ6);
2,5-bis((2-(pyrrolidin-l-yl)ethyl)amino)-1,4-benzoquinone (BQ9);
2,5-bis-(4-trifluoromethylbenzylamino)-1,4-benzoquinone (BQ10);
2,5-bis-(4-fluorobenzylamino)-1,4-benzoquinone (BQ11);
2,5-bis-(3,4-dimethoxybenzylamino)-1,4-benzoquinone (BQ12); and
2,5-bis-(3,5-ditrifluoromethylbenzylamino)-1,4-benzoquinone (BQ14).

4. A method of treating cancer in a mammal comprising administering to the mammal an amount of a compound of formula (I), as claimed in claim 1, or the pharmaceutically acceptable salts or solvates thereof.

5. The method as claimed in claim 4, wherein the mammal is in need of cancer treatment.

6. The method as claimed in claim 4, wherein the mammal is a human.

7. The method as claimed in claim 4, wherein the method is for the treatment of pancreatic cancer, lung cancer, or breast cancer.

8. A method of stabilizing G-quadruplex DNA that may be formed in a telomere region of a mammalian chromosome, the method comprising binding a compound of formula (I), as claimed in claim 1, or the pharmaceutically acceptable salts or solvates thereof, to the G-quadruplex DNA.

9. The method as claimed in claim 8, wherein the mammalian chromosome is a human chromosome.

10. The method as claimed in claim 9, wherein the human chromosome is from a human in need of cancer treatment.

11. The method as claimed in claim 10, wherein the human is in need of cancer treatment for pancreatic cancer, lung cancer, or breast cancer.

12. A method of inhibit telomerase enzyme in a mammalian cell, particularly a human cell, comprising administering to the cell an amount of a compound of formula (I), as claimed in claim 1, or the pharmaceutically acceptable salts or solvates thereof

13. The method as claimed in claim 12, wherein the mammalian cell is from a mammal in need of cancer treatment.

14. The method as claimed in claim 13, wherein the mammal is in need of cancer treatment for pancreatic cancer, lung cancer, or breast cancer.

15. The method as claimed in claim 12, wherein the compound is administered in a therapeutically effective amount.

16. The method as claimed in claim 12, wherein the method comprises binding the compound of formula (I), or the pharmaceutically acceptable salts, or solvates thereof, to G-quadruplex DNA that may be formed in the mammalian cell or in the human cell.
